SEQUENCE LISTING (38 sequences)

SEQ ID NO: 1 - protein, 8 aa, Artificial Sequence
  Nb7 CDR1
AFTNAYMN

SEQ ID NO: 2 - protein, 8 aa, Artificial Sequence
  Nb7 CDR2
TRDSNRTS

SEQ ID NO: 3 - protein, 13 aa, Artificial Sequence
  Nb7 CDR3
AEPSGWWLDDDYW

SEQ ID NO: 4 - protein, 9 aa, Artificial Sequence
  7E12 CDR1
GYSITSGYY

SEQ ID NO: 5 - protein, 7 aa, Artificial Sequence
  7E12 CDR2
ISYDGNN

SEQ ID NO: 6 - protein, 12 aa, Artificial Sequence
  7E12 CDR3
ARGDYGSEAMDY

SEQ ID NO: 7 - protein, 8 aa, Artificial Sequence
  7G5 CDR1
GYTFTNYG

SEQ ID NO: 8 - protein, 8 aa, Artificial Sequence
  7G5 CDR2
INTYTGEP

SEQ ID NO: 9 - protein, 11 aa, Artificial Sequence
  7G5 CDR3
ARRSNPWPNDY

SEQ ID NO: 10 - protein, 8 aa, Artificial Sequence
  3C3 CDR1
GFNIRNTY

SEQ ID NO: 11 - protein, 8 aa, Artificial Sequence
  3C3 CDR2
IDPANGNT

SEQ ID NO: 12 - protein, 12 aa, Artificial Sequence
  3C3 CDR3
IMHAEGQGWFAY

SEQ ID NO: 13 - protein, 8 aa, Artificial Sequence
  7C6 CDR1
GYTFTSYY

SEQ ID NO: 14 - protein, 8 aa, Artificial Sequence
  7C6 CDR2
INTGSGGT

SEQ ID NO: 15 - protein, 18 aa, Artificial Sequence
  7C6 CDR3
AREGTFYSGDVGYWYFDF

SEQ ID NO: 16 - protein, 8 aa, Artificial Sequence
  7C7 CDR1
GFTFNKYW

SEQ ID NO: 17 - protein, 8 aa, Artificial Sequence
  7C7 CDR2
ITNSGGNT

SEQ ID NO: 18 - protein, 6 aa, Artificial Sequence
  7C7 CDR3
TLGVDY

SEQ ID NO: 19 - protein, 8 aa, Artificial Sequence
  7G1 CDR1
GLSLTSNS

SEQ ID NO: 20 - protein, 7 aa, Artificial Sequence
  7G1 CDR2
IWSNGGT

SEQ ID NO: 21 - protein, 14 aa, Artificial Sequence
  7G1 CDR3
ARYYDGSYYWYFDF

SEQ ID NO: 22 - protein, 8 aa, Artificial Sequence
  8G10 CDR1
GYTFTDYE

SEQ ID NO: 23 - protein, 8 aa, Artificial Sequence
  8G10 CDR2
IDPETGGT

SEQ ID NO: 24 - protein, 12 aa, Artificial Sequence
  8G10 CDR3
TRWSLRPYAMDY

SEQ ID NO: 25 - protein, 119 aa, Artificial Sequence
  7E12 Heavy Chain
DVHLQESGPGLVKPSQSLSLTCSVTGYSITSGYYWNWIRQFPGNKLEWMGYISYDGNNNYNPSLKNRISITRDTSKNQFFLKLNSVTTEDTATYYCARGDYGSEAMDYWGQGTSVTVSS

SEQ ID NO: 26 - protein, 111 aa, Artificial Sequence
  7E12 Light Chain
DIRVTQSPASLAVSLGQRATISCRASKSVSTSGYSYMNWYQQKPGQPPQLLIYLASNLESGVPARFSGSGSGTDFTLNIHPVEEEDAAIYYCQHSRELPWTFGGGTKLEIK

SEQ ID NO: 27 - protein, 118 aa, Artificial Sequence
  7G5 Heavy Chain
QIQLVQSGPELKKPGETVKISCKASGYTFTNYGMNWVKQAPEKGLKWMGWINTYTGEPTYADDFKGRFAFSLETSASTAYLQINNLKNEDTATYFCARRSNPWPNDYWGQGTSLTVSS

SEQ ID NO: 28 - protein, 107 aa, Artificial Sequence
  7G5 Light Chain
DIQMTQSPSSFSASLGERVSLTCRASQEISGYLSWLQQKPDGTIKRLIYAASTLDSGVPKRFSGSRSGSDYSLTISSLESEDFADYYCLQYASYPWTFGGGTKLEIK

SEQ ID NO: 29 - protein, 119 aa, Artificial Sequence
  3C3 Heavy Chain
EVKLQQSGAELGKPGASVKLSCKVSGFNIRNTYMHWVNQRPGKGLEWIGRIDPANGNTIYAEKFKSKATLTADTSSNTVYIQLSQLKSDDTAIYFCIMHAEGQGWFAYWGQGTLVTVSS

SEQ ID NO: 30 - protein, 107 aa, Artificial Sequence
  3C3 Light Chain
IVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWK

SEQ ID NO: 31 - protein, 125 aa, Artificial Sequence
  7C6 Heavy Chain
QVQLQQSGAELAKPGSSVKISCKASGYTFTSYYISWIKQTTGQGLEYIGYINTGSGGTNYNEKFKGKATLTVDKSSSTAFMQLSSLTPDDSAVYYCAREGTFYSGDVGYWYFDFWGPGSMVTVSS

SEQ ID NO: 32 - protein, 113 aa, Artificial Sequence
  7C7 Heavy Chain
EVQLVESGGGLVQPGRSLKLSCVASGFTFNKYWMSWTRQAPGKGLEWVASITNSGGNTYYPDSVKGRFTISRDNAQNTLYLQMNSLRSEDTATYYCTLGVDYWGQGAMVTVSS

SEQ ID NO: 33 - protein, 112 aa, Artificial Sequence
  7C7 Light Chain
DVVMTQTPPSLSVTIGQSVSISCKSSQSLVYSDGKTYLHWLLQSSGRSPKRLIYQVSNLASGVPDRFSGTGSQKDFTLKISRVEAKDLGVYYCAQTTHFPYTFGAGTKLEMK

SEQ ID NO: 34 - protein, 119 aa, Artificial Sequence
  7G1 Heavy Chain
QVQLKESGPGLVQPSQTLSLTCTVSGLSLTSNSVSWIRQPPGKGLEWMGAIWSNGGTDYNSAIKSRLSISRDTSKSQVFLKVNSLQTEDAMYFCARYYDGSYYWYFDFWGPGTMVTVSS

SEQ ID NO: 35 - protein, 107 aa, Artificial Sequence; identical to SEQ ID NO: 30
  7G1 Light Chain

SEQ ID NO: 36 - protein, 119 aa, Artificial Sequence
  8G10 Heavy Chain
QVQLQQSGAELVRPGASVTLSCKASGYTFTDYEMHWVKQTPVHGLEWIGAIDPETGGTAYNQKFKGKATLTADKSSSTAYMELRSLTSEDSAVYYCTRWSLRPYAMDYWGQGTSVTVSS

SEQ ID NO: 37 - protein, 112 aa, Artificial Sequence
  8G10 Light Chain
DVVMTHTPLSLPVSLGDQASISCRSSQSLVHNNGNTYLHWYLQKPGQSPKLLIYKVSNRFSGVPDRFSGSGSGTDFTLKISRVEAEDLGLYFCSQSTHVPWTFGGGTKLEIK

SEQ ID NO: 38 - protein, 120 aa, Artificial Sequence
  Nb7
QVQLQESGGGLVYPGGSLRLSCTASGFAFTNAYMNWVRQAPGKGAEWVSGITRDSNRTSYADSVKGRFTISRDNAKNTLYLQMDSLKSDDTALYYCNAEPSGWWLDDDYWGQGTQVTVSS

(19) United States
(12) Patent Application Publication
(10) Pub. No.: US 2024/0293499 A1
(43) Pub. Date: Sep. 5, 2024

(54) VISTA AGONISTS AND RELATED COMPOSITIONS AND METHODS

(71) Applicant: Sunnybrook Research Institute, Toronto (CA)

(72) Inventors: Jean Gariepy, Toronto (CA); Vivian Ma, Toronto (CA); Amanda Sparkes, Stouffville (CA); Esther Matus, York (CA)

(21) Appl. No.: 18/572,826

(22) PCT Filed: Jun. 24, 2022
(86) PCT No.: PCT/CA2022/051018

§ 371 Date: Dec. 21, 2023

Related U.S. Application Data
(60) Provisional application No. 63/215,076, filed on Jun. 25, 2021.

(51) Int. Cl. A61K 38/08; A61K 38/10; A61P 37/06

(52) U.S. Cl. CPC A61K 38/08; A61K 38/10; A61P 37/06

(57) ABSTRACT

Described herein is a VISTA agonist for inhibiting an immune response, wherein the VISTA agonist causes at least one of the following: an increase in IL-10 expression; a decrease in proliferation of CD4+ T cells; a decrease in proliferation of CD8+ T cells; a decrease in IL-2 expression; a decrease in IFN-γ expression; and a decrease in IL-17 expression.

### FIELD

The present invention relates to immune checkpoint regulation. In particular, the present invention relates to VISTA agonists as well as related compositions and methods.

### BACKGROUND

V-domain Ig suppressor of T cell activation (VISTA) is a type I transmembrane protein that functions as an immune checkpoint and is encoded by the C10orf54 gene. VISTA is approximately 50 kDa and belongs to the immunoglobulin superfamily and has one IgV domain. VISTA is part of the B7 family, is primarily expressed in white blood cells and its transcription is partially controlled by p53. There is evidence that VISTA can act as both a ligand and a receptor on T cells to inhibit T cell effector function and maintain peripheral tolerance. VISTA is produced at high levels in tumor-infiltrating lymphocytes, such as myeloid-derived suppressor cells and regulatory T cells, and its blockade with an antibody results in delayed tumor growth in mouse models of melanoma and squamous cell carcinoma.

### SUMMARY OF THE INVENTION

In accordance with an aspect, there is provided a VISTA agonist for inhibiting an immune response, wherein the VISTA agonist causes at least one of the following:
an increase in IL-10 expression;
a decrease in proliferation of CD4⁺ T cells;
a decrease in proliferation of CD8⁺ T cells;
a decrease in IL-2 expression;
a decrease in IFN-γ expression; and
a decrease in IL-17 expression.
In an aspect, the VISTA agonist causes an increase in IL-10 expression, a decrease in proliferation of CD4⁺ and/or CD8⁺ T cells, and a decrease in IL-2 expression.

In an aspect, the VISTA agonist comprises or consists of a polypeptide having the amino acid sequence of CDR1, CDR2, and/or CDR3 of Nb7, 7E12, 7G5, 3C3, 7C6, 7C7, 7G1, and/or 8G10:

| | CDR1 | CDR2 | CDR3 |
|---|---|---|---|
| Nb7 | AFTNAYMN | TRDSNRTS | AEPSGWWLDDDYW |
| | (SEQ ID NO: 1) | (SEQ ID NO: 2) | (SEQ ID NO: 3) |
| 7E12 | GYSITSGYY | ISYDGNN | ARGDYGSEAMDY |
| | (SEQ ID NO: 4) | (SEQ ID NO: 5) | (SEQ ID NO: 6) |
| 7G5 | GYTFTNYG | INTYTGEP | ARRSNPWPNDY |
| | (SEQ ID NO: 7) | (SEQ ID NO: 8) | (SEQ ID NO: 9) |
| 3C3 | GFNIRNTY | IDPANGNT | IMHAEGQGWFAY |
| | (SEQ ID NO: 10) | (SEQ ID NO: 11) | (SEQ ID NO: 12) |
| 7C6 | GYTFTSYY | INTGSGGT | AREGTFYSGDVGYWYFDF |
| | (SEQ ID NO: 13) | (SEQ ID NO: 14) | (SEQ ID NO: 15) |
| 7C7 | GFTFNKYW | ITNSGGNT | TLGVDY |
| | (SEQ ID NO: 16) | (SEQ ID NO: 17) | (SEQ ID NO: 18) |
| 7G1 | GLSLTSNS | IWSNGGT | ARYYDGSYYWYFDF |
| | (SEQ ID NO: 19) | (SEQ ID NO: 20) | (SEQ ID NO: 21) |
| 8G10 | GYTFTDYE | IDPETGGT | TRWSLRPYAMDY |
| | (SEQ ID NO: 22) | (SEQ ID NO: 23) | (SEQ ID NO: 24) |

or a functional variant or fragment thereof that binds to and agonizes VISTA.

In an aspect, the VISTA agonist comprises or consists of a polypeptide having the amino acid sequence of the heavy and/or light chain of 7E12, 7G5, 3C3, 7C6, 7C7, 7G1, and/or 8G10 and/or the amino acid sequence of Nb7:

7E12 Heavy Chain:
(SEQ ID NO: 25)
DVHLQESGPGLVKPSQSLSLTCSVTGYSITSGYYWNWIRQFPGNKLEWMGYISYDGNNNYNPSLKN
RISITRDTSKNQFFLKLNSVTTEDTATYYCARGDYGSEAMDYWGQGTSVTVSS
7E12 Light Chain:
(SEQ ID NO: 26)
DIRVTQSPASLAVSLGQRATISCRASKSVSTSGYSYMNWYQQKPGQPPQLLIYLASNLESGVPARFS
GSGSGTDFTLNIHPVEEEDAAIYYCQHSRELPWTFGGGTKLEIK
7G5 Heavy Chain:
(SEQ ID NO: 27)
QIQLVQSGPELKKPGETVKISCKASGYTFTNYGMNWVKQAPEKGLKWMGWINTYTGEPTYADDFKG
RFAFSLETSASTAYLQINNLKNEDTATYFCARRSNPWPNDYWGQGTSLTVSS
7G5 Light Chain:
(SEQ ID NO: 28)
DIQMTQSPSSFSASLGERVSLTCRASQEISGYLSWLQQKPDGTIKRLIYAASTLDSGVPKRFSGSRSG
SDYSLTISSLESEDFADYYCLQYASYPWTFGGGTKLEIK
3C3 Heavy Chain:
(SEQ ID NO: 29)
EVKLQQSGAELGKPGASVKLSCKVSGFNIRNTYMHWVNQRPGKGLEWIGRIDPANGNTIYAEKFKSK
ATLTADTSSNTVYIQLSQLKSDDTAIYFCIMHAEGQGWFAYWGQGTLVTVSS
3C3 Light Chain:
(SEQ ID NO: 30)
IVLTQSPASLAVSLGQRATISYRASKSVSTSGYSYMHWNQQKPGQPPRLLIYLVSNLESGVPARFSGS
GSGTDFTLNIHPVEEEDAATYYCQHIRELTRSEGGPSWK
7C6 Heavy Chain:
(SEQ ID NO: 31)
QVQLQQSGAELAKPGSSVKISCKASGYTFTSYYISWIKQTTGQGLEYIGYINTGSGGTNYNEKFKGKA
TLTVDKSSSTAFMQLSSLTPDDSAVYYCAREGTFYSGDVGYWYFDFWGPGSMVTVSS
7C7 Heavy Chain:
(SEQ ID NO: 32)
EVQLVESGGGLVQPGRSLKLSCVASGFTFNKYWMSWTRQAPGKGLEWVASITNSGGNTYYPDSVK
GRFTISRDNAQNTLYLQMNSLRSEDTATYYCTLGVDYWGQGAMVTVSS
7C7 Light Chain:
(SEQ ID NO: 33)
DVVMTQTPPSLSVTIGQSVSISCKSSQSLVYSDGKTYLHWLLQSSGRSPKRLIYQVSNLASGVPDRFS
GTGSQKDFTLKISRVEAKDLGVYYCAQTTHEPYTFGAGTKLEMK
7G1 Heavy Chain:
(SEQ ID NO: 34)
QVQLKESGPGLVQPSQTLSLTCTVSGLSLTSNSVSWIRQPPGKGLEWMGAIWSNGGTDYNSAIKSR
LSISRDTSKSQVFLKVNSLQTEDAMYFCARYYDGSYYWYFDFWGPGTMVTVSS
7G1 Light Chain:
(SEQ ID NO: 35)
IVLTQSPASLAVSLGQRATISYRASKSVSTSGYSYMHWNQQKPGQPPRLLIYLVSNLESGVPARFSGS
GSGTDFTLNIHPVEEEDAATYYCQHIRELTRSEGGPSWK
8G10 Heavy Chain:
(SEQ ID NO: 36)
QVQLQQSGAELVRPGASVTLSCKASGYTFTDYEMHWVKQTPVHGLEWIGAIDPETGGTAYNQKFKG
KATLTADKSSSTAYMELRSLTSEDSAVYYCTRWSLRPYAMDYWGQGTSVTVSS
8G10 Light Chain:
(SEQ ID NO: 37)
DVVMTHTPLSLPVSLGDQASISCRSSQSLVHNNGNTYLHWYLQKPGQSPKLLIYKVSNRFSGVPDRF
SGSGSGTDFTLKISRVEAEDLGLYFCSQSTHVPWTFGGGTKLEIK
Nb7:
(SEQ ID NO: 38)
QVQLQESGGGLVYPGGSLRLSCTASGFAFTNAYMNWVRQAPGKGAEWVSGITRDSNRTSYADSVK
GRFTISRDNAKNTLYLQMDSLKSDDTALYYCNAEPSGWWLDDDYWGQGTQVTVSS

or a functional variant or fragment thereof that binds to and agonizes VISTA.

In an aspect, the functional variant has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the recited amino acid sequence.

In an aspect, the VISTA agonist comprises or consists of a polypeptide having 100% identity to the recited amino acid sequence.

In accordance with an aspect, there is provided a polynucleotide encoding the VISTA agonist described herein.

In accordance with an aspect, there is provided a vector comprising the polynucleotide described herein.

In accordance with an aspect, there is provided a host cell comprising the vector described herein.

In accordance with an aspect, there is provided a composition comprising the VISTA agonist described herein and a carrier.

In an aspect, the VISTA agonist, polynucleotide, the vector, the host cell, or the composition described herein is for treating and/or preventing an inflammatory disorder.

In an aspect, the VISTA agonist, the polynucleotide, the vector, the host cell, or the composition described herein is for treating and/or preventing an autoimmune disease.

In accordance with an aspect, there is provided a method for inhibiting an immune response, the method comprising administering the VISTA agonist, the polynucleotide, the vector, the host cell, or the composition described herein.

In accordance with an aspect, there is provided a method for treating and/or preventing an inflammatory disorder, the method comprising administering the VISTA agonist, the polynucleotide, the vector, the host cell, or the composition described herein.

In accordance with an aspect, there is provided a method for treating and/or preventing an autoimmune disease, the method comprising administering the VISTA agonist, the polynucleotide, the vector, the host cell, or the composition described herein.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figure, in which:

FIG. 1. Sequence alignment and production of anti-VISTA mAbs and Nbs. (A) Sequence alignments of the variable regions of heavy chains of mouse anti-hVISTA mAbs, anti-hVISTA Nbs, and rat anti-hmVISTA mAbs, clustered based on sequence similarity of CDR3. (B) SDS-PAGE gel and Western blot of anti-VISTA mAbs and Nbs under reducing conditions. Bands on Western blots were detected using an antibody conjugated to HRP that recognizes heavy and light chains of mouse or rat IgGs.

FIG. 2. Binding of mAbs and Nbs to VISTA. (A-C) SPR single-cycle kinetics sensorgrams (in red) and fitted curves (in black) with equilibrium (KD), association (ka) and dissociation (kd) rate constants depicting the binding of (A) mouse anti-hVISTA mAbs (7E12, 7G5, 8G10) and anti-hVISTA Nbs (Nb1, Nb5, Nb7) to human VISTA, (B) rat anti-hmVISTA mAbs (3C3, 7C6, 7C7, 7G1) to human VISTA, and (C) rat anti-hmVISTA mAbs to mouse VISTA. (D) Competition ELISA of anti-hVISTA Nbs, or an irrelevant control Nb19, with representative anti-hVISTA (7E12) or anti-hmVISTA (7C6) mAbs (n=3).

FIG. 3. Binding of anti-VISTA mAbs and Nbs to immune cell sub-populations present in human PBMCs and mouse splenocytes. (A) Right panel histograms depict the binding of a commercial anti-hVISTA antibody (B7H5DS8), anti-hVISTA murine mAbs (7E12, 7G5, 8G10) and Nbs (Nb1, Nb5, Nb7), and anti-hmVISTA rat mAbs (3C3, 7C6, 7C7, 7G1) to human CD14+CD16+ monocytes and CD4+ and CD8+ T cells from PBMCs of 5 donors (reported as ΔMFI, the difference between MFI values recorded for mAbs or Nb and their respective isotype control). Left panel shows a representative cytometric profile of the binding of murine anti-hVISTA mAb 8G10 to human CD14+CD16-monocytes relative to its isotype control murine IgG (mIgG), normalized to mode (% of counts in the maximum peak). (B) Histograms depicting the binding of anti-hmVISTA rat mAbs to macrophages, neutrophils, and CD4+ and CD8+ T cells from splenocytes of 3 mice (reported as ΔMFI). Data is shown as mean±SD. *p<0.05 by Student's t-test between MFIs of mAbs or Nbs and their isotype controls.

FIG. 4: Effect of anti-hVISTA mAbs and Nb7 and anti-hmVISTA mAbs on T cell proliferation in human PBMC cultures. Percentages of proliferated (A) CD4+ and (B) CD8+ T cells in human PBMC cultures after stimulation with 1 μg/mL ConA and anti-hVISTA mouse mAbs (7E12, 7G5, 8G10) and Nb7, or anti-hmVISTA rat mAbs (3C3, 7C6, 7C7, 7G1) for 4 days. Representative CFSE T cell proliferation profiles are shown on the left side for ConA alone or in the presence of mAb 7E12 (A) or 8G10 (B), normalized to mode (% of counts in the maximum peak). Experiments were performed in replicates on 5 donors. Grey lines represent a separate paired experiment; black lines represent the average of all experiments.

FIG. 5. Effect of anti-hVISTA mAbs, Nb7, and anti-hmVISTA mAbs on cytokine production in human PBMC cultures. Concentrations of (A) IL-2, (B) IFNγ, and (C) IL-10 in human PBMC cultures after stimulation with 1 μg/mL ConA and anti-hVISTA murine mAbs (7E12, 7G5, 8G10), anti-hVISTA Nb7, or anti-hmVISTA rat mAbs (3C3, 7C6, 7C7, 7G1) for 2 days. Experiments were performed on 5 donors. Grey lines represent a separate paired experiment; black lines represent the average of all experiments.

FIG. 6. Effect of anti-hmVISTA rat mAbs T cell proliferation in mouse splenocyte cultures. Percentages of proliferated (A) CD4+ and (B) CD8+ T cells in mouse splenocyte cultures after stimulation with 1 μg/mL ConA and anti-hmVISTA rat mAbs for 3 days. Representative CFSE T cell proliferation profiles are shown on the left side for ConA alone or in the presence of mAb 7G1, normalized to mode (% of counts in the maximum peak). Each grey line represents a biological replicate (separate paired experiment performed on a different mice); black lines represent the average of all experiments.

FIG. 7. Effect of rat anti-hmVISTA mAbs on the inflammatory and immune status of mice treated topically with Imiquimod (IMQ). IMQ cream or Vaseline was topically applied daily to shaved areas on the back of female C57BL/6 mice. IMQ-treated mice received intraperitoneal injections of rat anti-hmVISTA mAbs, rIgG, or PBS every other day. (A) Graph showing the cumulative score representing the severity of psoriasis-like skin inflammation (erythema, scaling, and thickness, each out of 4; average of 3 independent scorers), with adjusted p-values comparing each group (n=5) to IMQ alone. (B) RNA expression of IL-17 in treated skin samples of mice sacrificed on day 3. (C) RNA expression of IFNγ in skin samples recovered from mice sacrificed on day 6. (D) Percentage of CD4+ and CD8+ T cells in splenocytes harvested from mice sacrificed on day 6, as determined by flow cytometry. Data is shown as mean±SD, each dot represents a mouse in the experimental group. *p<0.05, **p<0.01, ***p<0.001, ****p<0.0001 relative to IMQ-only groups by Student's t-test adjusted for multiple comparisons.

FIG. 8. SPR single-cycle kinetics sensorgrams (in red) and fitted curves (in black) depicting the absence of binding of mouse anti-hVISTA mAbs (7E12, 7G5, 8G10) and anti-hVISTA Nbs (Nb1, Nb5, Nb7) to murine VISTA.

### DETAILED DESCRIPTION

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only and is not intended to be limiting. Many patent applications, patents, and publications may be referred to herein to assist in understanding the aspects described. Each of these references is incorporated herein by reference in its entirety.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight or volume, typically less than 3% by weight, more typically less than 1%, and even more typically less than 0.1% by weight of non-specified component(s).

It will be understood that any component defined herein as being included may be explicitly excluded from the claimed invention by way of proviso or negative limitation.

In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

Although methods and materials similar or equivalent to those described herein can be used in the practice or testing of this disclosure, suitable methods and materials are described below. The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, typically, a human.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “antibody”, also referred to in the art as “immunoglobulin” (Ig), used herein refers to a protein constructed from paired heavy and light polypeptide chains; various Ig isotypes exist, including IgA, IgD, IgE, IgG, such as IgG₁, IgG₂, IgG₃, and IgG₄, and IgM. It will be understood that the antibody may be from any species, including human, mouse, rat, monkey, llama, or shark. When an antibody is correctly folded, each chain folds into a number of distinct globular domains joined by more linear polypeptide sequences. For example, in the case of IgGs, the immunoglobulin light chain folds into a variable (V_{L}) and a constant (CL) domain, while the heavy chain folds into a variable (V_{H}) and three constant (C_{H}, C_{H2}, C_{H3}) domains. Interaction of the heavy and light chain variable domains (V_{H} and V_{L}) results in the formation of an antigen binding region (Fv). Each domain has a well-established structure familiar to those of skill in the art.

The light and heavy chain variable regions are responsible for binding the target antigen and can therefore show significant sequence diversity between antibodies. The constant regions show less sequence diversity, and are responsible for binding a number of natural proteins to elicit important immunological events. The variable region of an antibody contains the antigen binding determinants of the molecule, and thus determines the specificity of an antibody for its target antigen. The majority of sequence variability occurs in six hypervariable regions, three each per variable heavy and light chain; the hypervariable regions combine to form the antigen-binding site, and contribute to binding and recognition of an antigenic determinant. The specificity and affinity of an antibody for its antigen is determined by the structure of the hypervariable regions, as well as their size, shape and chemistry of the surface they present to the antigen.

An “antibody fragment” as referred to herein may include any suitable antigen-binding antibody fragment known in the art. The antibody fragment may be a naturally-occurring antibody fragment, or may be obtained by manipulation of a naturally-occurring antibody or by using recombinant methods. For example, an antibody fragment may include, but is not limited to a Fv, single-chain Fv (scFv; a molecule consisting of V_{L} and V_{H} connected with a peptide linker), Fc, single-chain Fc, Fab, single-chain Fab, F(ab′)₂, single domain antibody (sdAb; a fragment composed of a single V_{L} or V_{H}), and multivalent presentations of any of these.

By the term “synthetic antibody” as used herein, is meant an antibody which is generated using recombinant DNA technology. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “epitope” refers to an antigenic determinant. An epitope is the particular chemical groups or peptide sequences on a molecule that are antigenic, that is, that elicit a specific immune response. An antibody specifically binds a particular antigenic epitope, e.g., on a polypeptide. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, about 11, or about 8 to about 12 amino acids in a unique spatial conformation. Methods of determining spatial conformation of epitopes include, for example, x-ray crystallography and 2-dimensional nuclear magnetic resonance. See, e.g., “Epitope Mapping Protocols” in Methods in Molecular Biology, Vol. 66, Glenn E. Morris, Ed (1996).

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the aspects described herein include, but are not limited to, the use of partial nucleotide sequences of more than one gene and that these nucleotide sequences could be arranged in various combinations to elicit the desired immune response. Moreover, a skilled artisan will understand that an antigen need not be encoded by a “gene” at all. It is readily apparent that an antigen can be synthesized or can be derived from a biological sample. Such a biological sample can include, but is not limited to a tissue sample, a cell, or a biological fluid.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Variants” are biologically active constructs, antibodies, or fragments thereof having an amino acid sequence that differs from a comparator sequence by virtue of an insertion, deletion, modification and/or substitution of one or more amino acid residues within the comparative sequence. Variants generally have less than 100% sequence identity with the comparative sequence. Ordinarily, however, a biologically active variant will have an amino acid sequence with at least about 70% amino acid sequence identity with the comparative sequence, such as at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity. The variants include peptide fragments, for example of at least 10 amino acids, that retain some level of the biological activity of the comparator sequence. Variants also include polypeptides wherein one or more amino acid residues are added at the N- or C-terminus of, or within, the comparative sequence. Variants also include polypeptides where a number of amino acid residues are deleted and optionally substituted by one or more amino acid residues. Variants also may be covalently modified, for example by substitution with a moiety other than a naturally occurring amino acid or by modifying an amino acid residue to produce a non-naturally occurring amino acid.

“Percent amino acid sequence identity” is defined herein as the percentage of amino acid residues in the candidate sequence that are identical with the residues in the sequence of interest, such as the polypeptides of the invention, after aligning the sequences and introducing gaps, if necessary, to achieve the maximum percent sequence identity, and not considering any conservative substitutions as part of the sequence identity. None of N-terminal, C-terminal, or internal extensions, deletions or insertions into the candidate sequence shall be construed as affecting sequence identity or homology. Methods and computer programs for the alignment are well known in the art, such as “BLAST”.

The constructs described herein may include modifications. Such modifications include, but are not limited to, conjugation to an effector molecule. Modifications further include, but are not limited to conjugation to detectable reporter moieties. Modifications that extend half-life (e.g., pegylation) are also included. Proteins and non-protein agents may be conjugated to the constructs by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990), which is incorporated by reference herein and those described by Amon et al., Adv. Exp. Med. Biol. 303, 79-90 (1991) and by Kiseleva et al, Mol. Biol. (USSR)25, 508-514 (1991), both of which are incorporated by reference herein.

“Active” or “activity” for the purposes herein refers to a biological and/or an immunological activity of the polypeptides or compositions described herein, wherein “biological” activity refers to a biological function (either inhibitory or stimulatory) caused by the polypeptides or compositions.

The terms “therapeutically effective amount”, “effective amount” or “sufficient amount” mean a quantity sufficient, when administered to a subject, including a mammal, for example a human, to achieve a desired result, for example an amount effective to inhibit an immune response. Effective amounts of the polypeptides described herein may vary according to factors such as the condition in question, age, sex, and weight of the subject. Dosage or treatment regimes may be adjusted to provide the optimum therapeutic response, as is understood by a skilled person. For example, administration of a therapeutically effective amount of the polypeptides described herein is, in aspects, sufficient to inhibit an immune response. In aspects, the inhibition of the immune response may be measured by increases or decreases in relevant cytokine expression and/or by decreases in T cell proliferation. In another example, administration of a therapeutically effective amount of the polypeptides described herein is, in aspects, sufficient to treat and/or prevent an inflammatory condition and/or an autoimmune disease.

Moreover, a treatment regime of a subject with a therapeutically effective amount may consist of a single administration, or alternatively comprise a series of applications. The length of the treatment period depends on a variety of factors, such as the condition in question, the age of the subject, the concentration of the agent, the responsiveness of the patient to the agent, or a combination thereof. It will also be appreciated that the effective dosage of the agent used for the treatment may increase or decrease over the course of a particular treatment regime. Changes in dosage may result and become apparent by standard diagnostic assays known in the art. The polypeptides described herein may, in aspects, be administered before, during or after treatment with conventional therapies for the disease or disorder in question, such as an inflammatory condition and/or an autoimmune disease.

The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

Administration “in combination with” one or more further therapeutic agents includes simultaneous (concurrent) and consecutive administration in any order.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration.

The term “pharmaceutically acceptable carrier” includes, but is not limited to solvents, dispersion media, coatings, antibacterial agents, antifungal agents, isotonic and/or absorption delaying agents and the like. The use of pharmaceutically acceptable carriers is well known.

### VISTA Agonists

Described herein are VISTA agonists that inhibit immune responses. The inhibition of an immune response can be measured in many different ways and is typically measured through an increase or decrease in relevant cytokines and/or through a decrease in T cell proliferation.

For example, typically the VISTA agonists described herein cause an increase in IL-10 expression, a decrease in proliferation of CD4⁺ T cells, a decrease in proliferation of CD8⁺ T cells, a decrease in IL-2 expression, a decrease in IFN-γ expression, and/or a decrease in IL-17 expression. For example, typically, the VISTA agonists at least cause an increase in IL-10 expression, a decrease in proliferation of CD4⁺ and/or CD8⁺ T cells, and a decrease in IL-2 expression.

The VISTA agonists are typically antibodies or antibody-like molecules such as nanobodies. A number of exemplary antibodies were prepared and tested as described herein and other antibodies with similar binding and effector functions are also encompassed herein. For example, antibodies 7E12, 7G5, 3C3, 7C6, 7C7, and 7G1 and nanobody Nb7 were prepared and found to be VISTA agonists and 8G10 was found to be a weak antagonist. Other antibodies comprising one or more of the CDRs of these antibodies are contemplated.

In a specific example, the VISTA agonists described herein comprise or consist of a polypeptide having the amino acid sequence of CDR1, CDR2, and/or CDR3 of Nb7, 7E12, 7G5, 3C3, 7C6, 7C7, 7G1, and/or 8G10:

| | CDR1 | CDR2 | CDR3 |
|---|---|---|---|
| Nb7 | AFTNAYMN | TRDSNRTS | AEPSGWWLDDDYW |
| | (SEQ ID NO: 1) | (SEQ ID NO: 2) | (SEQ ID NO: 3) |
| 7E12 | GYSITSGYY | ISYDGNN | ARGDYGSEAMDY |
| | (SEQ ID NO: 4) | (SEQ ID NO: 5) | (SEQ ID NO: 6) |
| 7G5 | GYTFTNYG | INTYTGEP | ARRSNPWPNDY |
| | (SEQ ID NO: 7) | (SEQ ID NO: 8) | (SEQ ID NO: 9) |
| 3C3 | GFNIRNTY | IDPANGNT | IMHAEGQGWFAY |
| | (SEQ ID NO: 10) | (SEQ ID NO: 11) | (SEQ ID NO: 12) |
| 7C6 | GYTFTSYY | INTGSGGT | AREGTFYSGDVGYWYFDF |
| | (SEQ ID NO: 13) | (SEQ ID NO: 14) | (SEQ ID NO: 15) |
| 7C7 | GFTFNKYW | ITNSGGNT | TLGVDY |
| | (SEQ ID NO: 16) | (SEQ ID NO: 17) | (SEQ ID NO: 18) |
| 7G1 | GLSLTSNS | IWSNGGT | ARYYDGSYYWYFDF |
| | (SEQ ID NO: 19) | (SEQ ID NO: 20) | (SEQ ID NO: 21) |
| 8G10 | GYTFTDYE | IDPETGGT | TRWSLRPYAMDY |
| | (SEQ ID NO: 22) | (SEQ ID NO: 23) | (SEQ ID NO: 24) |

Functional variants or fragments of these sequences that bind to and agonize VISTA are also contemplated for use herein.

In another specific example, the VISTA agonists described herein comprise or consist of a polypeptide having the amino acid sequence of the heavy and/or light chain of 7E12, 7G5, 303, 7C6, 7C7, 7G1, and/or 8G10 and/or the amino acid sequence of Nb7:

7E12 Heavy Chain:
(SEQ ID NO: 25)
DVHLQESGPGLVKPSQSLSLTCSVTGYSITSGYYWNWIRQFPGNKLEWMGYISYDGNNNYNPSLKN
RISITRDTSKNQFFLKLNSVTTEDTATYYCARGDYGSEAMDYWGQGTSVTVSS
7E12 Light Chain:
(SEQ ID NO: 26)
DIRVTQSPASLAVSLGQRATISCRASKSVSTSGYSYMNWYQQKPGQPPQLLIYLASNLESGVPARFS
GSGSGTDFTLNIHPVEEEDAAIYYCQHSRELPWTFGGGTKLEIK
7G5 Heavy Chain:
(SEQ ID NO: 27)
QIQLVQSGPELKKPGETVKISCKASGYTFTNYGMNWVKQAPEKGLKWMGWINTYTGEPTYADDFKG
RFAFSLETSASTAYLQINNLKNEDTATYFCARRSNPWPNDYWGQGTSLTVSS
7G5 Light Chain:
(SEQ ID NO: 28)
DIQMTQSPSSFSASLGERVSLTCRASQEISGYLSWLQQKPDGTIKRLIYAASTLDSGVPKRFSGSRSG
SDYSLTISSLESEDFADYYCLQYASYPWTFGGGTKLEIK
3C3 Heavy Chain:
(SEQ ID NO: 29)
EVKLQQSGAELGKPGASVKLSCKVSGFNIRNTYMHWVNQRPGKGLEWIGRIDPANGNTIYAEKFKSK
ATLTADTSSNTVYIQLSQLKSDDTAIYFCIMHAEGQGWFAYWGQGTLVTVSS
3C3 Light Chain:
(SEQ ID NO: 30)
IVLTQSPASLAVSLGQRATISYRASKSVSTSGYSYMHWNQQKPGQPPRLLIYLVSNLESGVPARFSGS
GSGTDFTLNIHPVEEEDAATYYCQHIRELTRSEGGPSWK
7C6 Heavy Chain:
(SEQ ID NO: 31)
QVQLQQSGAELAKPGSSVKISCKASGYTFTSYYISWIKQTTGQGLEYIGYINTGSGGTNYNEKFKGKA
TLTVDKSSSTAFMQLSSLTPDDSAVYYCAREGTFYSGDVGYWYFDFWGPGSMVTVSS
7C7 Heavy Chain:
(SEQ ID NO: 32)
EVQLVESGGGLVQPGRSLKLSCVASGFTFNKYWMSWTRQAPGKGLEWVASITNSGGNTYYPDSVK
GRFTISRDNAQNTLYLQMNSLRSEDTATYYCTLGVDYWGQGAMVTVSS
7C7 Light Chain:
(SEQ ID NO: 33)
DVVMTQTPPSLSVTIGQSVSISCKSSQSLVYSDGKTYLHWLLQSSGRSPKRLIYQVSNLASGVPDRFS
GTGSQKDFTLKISRVEAKDLGVYYCAQTTHEPYTFGAGTKLEMK
7G1 Heavy Chain:
(SEQ ID NO: 34)
QVQLKESGPGLVQPSQTLSLTCTVSGLSLTSNSVSWIRQPPGKGLEWMGAIWSNGGTDYNSAIKSR
LSISRDTSKSQVFLKVNSLQTEDAMYFCARYYDGSYYWYFDFWGPGTMVTVSS
7G1 Light Chain:
(SEQ ID NO: 35)
IVLTQSPASLAVSLGQRATISYRASKSVSTSGYSYMHWNQQKPGQPPRLLIYLVSNLESGVPARFSGS
GSGTDFTLNIHPVEEEDAATYYCQHIRELTRSEGGPSWK
8G10 Heavy Chain:
(SEQ ID NO: 36)
QVQLQQSGAELVRPGASVTLSCKASGYTFTDYEMHWVKQTPVHGLEWIGAIDPETGGTAYNQKFKG
KATLTADKSSSTAYMELRSLTSEDSAVYYCTRWSLRPYAMDYWGQGTSVTVSS
8G10 Light Chain:
(SEQ ID NO: 37)
DVVMTHTPLSLPVSLGDQASISCRSSQSLVHNNGNTYLHWYLQKPGQSPKLLIYKVSNRFSGVPDRF
SGSGSGTDFTLKISRVEAEDLGLYFCSQSTHVPWTFGGGTKLEIK
Nb7:
(SEQ ID NO: 38)
QVQLQESGGGLVYPGGSLRLSCTASGFAFTNAYMNWVRQAPGKGAEWVSGITRDSNRTSYADSVK
GRFTISRDNAKNTLYLQMDSLKSDDTALYYCNAEPSGWWLDDDYWGQGTQVTVSS

Functional variants or fragments of these sequences that bind to and agonize VISTA are also contemplated for use herein.

Sequences that are substantially identical to the above sequences are also contemplated, such as those that are at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical. Fragments of the sequences or the substantially identical variant sequences are also contemplated herein.

A substantially identical sequence may comprise one or more conservative amino acid mutations. It is known in the art that one or more conservative amino acid mutations to a reference sequence may yield a mutant peptide with no substantial change in physiological, chemical, or functional properties compared to the reference sequence; in such a case, the reference and mutant sequences would be considered “substantially identical” polypeptides. Conservative amino acid mutation may include addition, deletion, or substitution of an amino acid; a conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (Ille or I), phenylalanine (Phe or F), valine (Val or V), leucine (Leu or L), tryptophan (Trp or W), methionine (Met or M), alanine (Ala or A), and glycine (Gly or G).

“Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), BLAST-P, Blast-N, or FASTA-N, or any other appropriate software that is known in the art.

The substantially identical sequences of the present invention may be at least 85% identical; in another example, the substantially identical sequences may be at least 70, 75, 80, 85, 90, 95, 96, 97, 98, 99, or 100% (or any percentage there between) identical at the amino acid level to sequences described herein. In specific aspects, the substantially identical sequences retain the activity and specificity of the reference sequence. In a non-limiting embodiment, the difference in sequence identity may be due to conservative amino acid mutation(s).

The constructs described herein may also comprise additional sequences to aid in their expression, detection or purification. Any such sequences or tags known to those of skill in the art may be used. For example, and without wishing to be limiting, the constructs may comprise a targeting or signal sequence (for example, but not limited to ompA), a detection tag, exemplary tag cassettes include Strep tag, or any variant thereof; see, e.g., U.S. Pat. No. 7,981,632, His tag, Flag tag having the sequence motif DYKDDDDK, Xpress tag, Avi tag, Calmodulin tag, Polyglutamate tag, HA tag, Myc tag, Nus tag, S tag, SBP tag, Softag 1, Softag 3, V5 tag, CREB-binding protein (CBP), glutathione S-transferase (GST), maltose binding protein (MBP), green fluorescent protein (GFP), Thioredoxin tag, or any combination thereof; a purification tag (for example, but not limited to a Hiss or His₆), or a combination thereof.

In another example, the additional sequence may be a biotin recognition site such as that described by Cronan et al in WO 95/04069 or Voges et al in WO/2004/076670. As is also known to those of skill in the art, linker sequences may be used in conjunction with the additional sequences or tags.

More specifically, a tag cassette may comprise an extracellular component that can specifically bind to an antibody with high affinity or avidity. Within a single chain fusion protein structure, a tag cassette may be located (a) immediately amino-terminal to a connector region, (b) interposed between and connecting linker modules, (c) immediately carboxy-terminal to a binding domain, (d) interposed between and connecting a binding domain (e.g., scFv or scFab) to an effector domain, (e) interposed between and connecting subunits of a binding domain, or (f) at the amino-terminus of a single chain fusion protein. In certain embodiments, one or more junction amino acids may be disposed between and connecting a tag cassette with a hydrophobic portion, or disposed between and connecting a tag cassette with a connector region, or disposed between and connecting a tag cassette with a linker module, or disposed between and connecting a tag cassette with a binding domain.

Also encompassed herein are isolated or purified polypeptides, or fragments thereof immobilized onto a surface using various methodologies; for example, and without wishing to be limiting, the polypeptides may be linked or coupled to the surface via His-tag coupling, biotin binding, covalent binding, adsorption, and the like. The solid surface may be any suitable surface, for example, but not limited to the well surface of a microtiter plate, channels of surface plasmon resonance (SPR) sensorchips, membranes, beads (such as magnetic-based or sepharose-based beads or other chromatography resin), glass, a film, or any other useful surface.

In other aspects, the constructs may be linked to a cargo molecule; the constructs may deliver the cargo molecule to a desired site and may be linked to the cargo molecule using any method known in the art (recombinant technology, chemical conjugation, chelation, etc.). The cargo molecule may be any type of molecule, such as a therapeutic or diagnostic agent. For example, and without wishing to be limiting in any manner, the therapeutic agent may be a radioisotope, which may be used for radioimmunotherapy; a toxin, such as an immunotoxin; a cytokine, such as an immunocytokine; a cytotoxin; an apoptosis inducer; an enzyme; an anti-cancer antibody for immunotherapy; or any other suitable therapeutic molecule known in the art. In the alternative, a diagnostic agent may include, but is by no means limited to a radioisotope, a paramagnetic label such as gadolinium or iron oxide, a fluorophore, a Near Infra-Red (NIR) fluorochrome or dye (such as Cy3, Cy5.5, Alexa680, Dylight680, or Dylight800), an affinity label (for example biotin, avidin, etc), fused to a detectable protein-based molecule, or any other suitable agent that may be detected by imaging methods. In a specific, non-limiting example, the construct may be linked to a fluorescent agent such as FITC or may genetically be fused to the Enhanced Green Fluorescent Protein (EGFP).

The polypeptides described herein specifically bind to their targets. Antibody specificity, which refers to selective recognition of an antibody for a particular epitope of an antigen, of the antibodies or fragments described herein can be determined based on affinity and/or avidity. Affinity, represented by the equilibrium constant for the dissociation of an antigen with an antibody (K_{D}), measures the binding strength between an antigenic determinant (epitope) and an antibody binding site. Avidity is the measure of the strength of binding between an antibody with its antigen. Antibodies typically bind with a K_{D} of 10⁻⁵ to 10⁻¹¹ M. Any K_{D} greater than 10⁻⁴ M is generally considered to indicate non-specific binding. The lesser the value of the K_{D}, the stronger the binding strength between an antigenic determinant and the antibody binding site. In aspects, the antibodies described herein have a K_{D} of less than 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M.

Also described herein are nucleic acid molecules encoding the constructs described herein, as well as vectors comprising the nucleic acid molecules and host cells comprising the vectors.

Polynucleotides encoding the constructs described herein include polynucleotides with nucleic acid sequences that are substantially the same as the nucleic acid sequences of the polynucleotides of the present invention. “Substantially the same” nucleic acid sequence is defined herein as a sequence with at least 70%, at least 75%, at least 80%, at least 85%, at least 90%, at least 91%, at least 92%, at least 93%, at least 94%, at least 95% identity to another nucleic acid sequence when the two sequences are optimally aligned (with appropriate nucleotide insertions or deletions) and compared to determine exact matches of nucleotides between the two sequences.

Suitable sources of polynucleotides that encode fragments of antibodies include any cell, such as hybridomas and spleen cells, that express the full-length antibody. The fragments may be used by themselves as antibody equivalents, or may be recombined into equivalents, as described above. The DNA deletions and recombinations described in this section may be carried out by known methods, such as those described in the published patent applications listed above in the section entitled “Functional Equivalents of Antibodies” and/or other standard recombinant DNA techniques, such as those described below. Another source of DNAs are single chain antibodies produced from a phage display library, as is known in the art.

Additionally, expression vectors are provided containing the polynucleotide sequences previously described operably linked to an expression sequence, a promoter and an enhancer sequence. A variety of expression vectors for the efficient synthesis of antibody polypeptide in prokaryotic, such as bacteria and eukaryotic systems, including but not limited to yeast and mammalian cell culture systems have been developed. The vectors of the present invention can comprise segments of chromosomal, non-chromosomal and synthetic DNA sequences.

Any suitable expression vector can be used. For example, prokaryotic cloning vectors include plasmids from E. coli, such as colE1, pCRI, pBR322, pMB9, pUC, pKSM, and RP4. Prokaryotic vectors also include derivatives of phage DNA such as MI3 and other filamentous single-stranded DNA phages. An example of a vector useful in yeast is the 2μ plasmid. Suitable vectors for expression in mammalian cells include well-known derivatives of SV-40, adenovirus, retrovirus-derived DNA sequences and shuttle vectors derived from combination of functional mammalian vectors, such as those described above, and functional plasmids and phage DNA.

Additional eukaryotic expression vectors are known in the art (e.g., P J. Southern & P. Berg, J. Mol. Appl. Genet, 1:327-341 (1982); Subramani et al, Mol. Cell. Biol, 1: 854-864 (1981); Kaufinann & Sharp, “Amplification And Expression of Sequences Cotransfected with a Modular Dihydrofolate Reductase Complementary DNA Gene,” J. Mol. Biol, 159:601-621 (1982); Kaufhiann & Sharp, Mol. Cell. Biol, 159:601-664 (1982); Scahill et al., “Expression And Characterization Of The Product Of A Human Immune Interferon DNA Gene In Chinese Hamster Ovary Cells,” Proc. Nat'l Acad. Sci USA, 80:4654-4659 (1983); Urlaub & Chasin, Proc. Nat'l Acad. Sci USA, 77:4216-4220, (1980), all of which are incorporated by reference herein).

The expression vectors typically contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Also described herein are recombinant host cells containing the expression vectors previously described. The constructs described herein can be expressed in cell lines other than in hybridomas. Nucleic acids, which comprise a sequence encoding a polypeptide, can be used for transformation of a suitable mammalian host cell.

Cell lines of particular preference are selected based on high level of expression, constitutive expression of protein of interest and minimal contamination from host proteins. Mammalian cell lines available as hosts for expression are well known in the art and include many immortalized cell lines, such as but not limited to, Chinese Hamster Ovary (CHO) cells, Baby Hamster Kidney (BHK) cells and many others. Suitable additional eukaryotic cells include yeast and other fungi. Useful prokaryotic hosts include, for example, E. coli, such as E. coli SG-936, E. coli HB 101, E. coli W3110, E. coli X1776, E. coli X2282, E. coli DHI, and E. coli MRC1, Pseudomonas, Bacillus, such as Bacillus subtilis, and Streptomyces.

These present recombinant host cells can be used to produce proteins by culturing the cells under conditions permitting expression of the polypeptide and purifying the polypeptide from the host cell or medium surrounding the host cell. Targeting of the expressed polypeptide for secretion in the recombinant host cells can be facilitated by inserting a signal or secretory leader peptide-encoding sequence (See, Shokri et al, (2003) Appl Microbiol Biotechnol. 60(6): 654-664, Nielsen et al, Prot. Eng., 10:1-6 (1997); von Heinje et al., Nucl. Acids Res., 14:4683-4690 (1986), all of which are incorporated by reference herein) at the 5′ end of the antibody-encoding gene of interest. These secretory leader peptide elements can be derived from either prokaryotic or eukaryotic sequences. Accordingly suitably, secretory leader peptides are used, being amino acids joined to the N-terminal end of a polypeptide to direct movement of the polypeptide out of the host cell cytosol and secretion into the medium.

The constructs described herein can be fused to additional amino acid residues. Such amino acid residues can be a peptide tag to facilitate isolation, for example. Other amino acid residues for homing of the antibodies to specific organs or tissues are also contemplated.

Also described herein are methods for inhibiting an immune response, methods for treating and/or preventing an inflammatory disorder, and methods of treating and/or preventing an autoimmune disease. The methods comprise administering the polypeptides, polynucleotides, vectors, cells, or compositions described herein to a subject in need thereof.

The polypeptides, polynucleotides, vectors, cells, or compositions described herein can be used in any amount, for example from about 0.0001 μg/ml to about 10000 μg/ml, such as from about 0.0001, about 0.001, about 0.01, about 0.1, about 1, about 10, about 100, or about 1000 to about 0.001, about 0.01, about 0.1, about 1, about 10, about 100, about 1000, or about 10000 μg/ml. In other aspects, the polypeptides, polynucleotides, vectors, cells, or compositions may be used in amounts of from about 0.0001 UM to about 10000 UM, such as from about 0.0001, about 0.001, about 0.01, about 0.1, about 1, about 10, about 100, or about 1000 to about 0.001, about 0.01, about 0.1, about 1, about 10, about 100, about 1000, or about 10000 μM. In other aspects, the polypeptides, polynucleotides, vectors, cells, or compositions may be used in amounts of from about 0.0001 μg/kg or mg/kg to about 10000 μg/kg or mg/kg, such as from about 0.0001, about 0.001, about 0.01, about 0.1, about 1, about 10, about 100, or about 1000 to about 0.001, about 0.01, about 0.1, about 1, about 10, about 100, about 1000, or about 10000 μg/kg or mg/kg.

Any suitable method or route can be used to administer the polypeptides described herein. Routes of administration include, for example, oral, intravenous, intraperitoneal, subcutaneous, or intramuscular administration.

It is understood that polypeptides described herein, where used in a mammal for the purpose of prophylaxis or treatment, will typically be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection may, as is well known in the art, be formulated so as to provide quick, sustained or delayed release of the active ingredient after administration to the mammal.

Although human antibodies are particularly useful for administration to humans, they may be administered to other mammals as well. The term “mammal” as used herein is intended to include, but is not limited to, humans, laboratory animals, domestic pets and farm animals.

The following examples do not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989), Molecular Cloning: A Laboratory Manual, 2nd edition, Cold Spring Harbor Laboratory Press, which is incorporated by reference herein.

Without further description, it is believed that one of ordinary skill in the art can, using the preceding description and the following illustrative examples, make and utilize the compounds of the present invention and practice the claimed methods. The following working examples therefore, specifically point out the typical aspects of the present invention and are not to be construed as limiting in any way in the remainder of the disclosure.

### EXAMPLES

### Example 1: Agonistic Nanobodies and Antibodies to Human VISTA

### Abstract

The V-domain Ig Suppressor of T-cell Activation (VISTA) is an immune checkpoint regulator that negatively suppresses immune responses and is readily expressed on human and murine myeloid cells and T cells. This immunosuppressive pathway can be activated using VISTA agonists. Here, we report the development of murine anti-human VISTA specific (anti-hVISTA) monoclonal antibodies (mAbs), anti-hVISTA nanobodies (Nbs), and cross-reactive rat anti-murine/human VISTA (anti-hmVISTA) mAbs. All mAbs and Nbs generated bound to VISTA of reactive species (human or murine) with dissociation constants in the sub-nanomolar or low nanomolar range. Competition analysis revealed that the selected Nbs were binding to the same, or to nearby, epitope(s) as the human VISTA-specific mAbs. However, the cross-reactive mAbs only partially competed with Nbs for binding to hVISTA. All mAbs and one Nb (hVISTANb7) were able to strongly detect VISTA expression on primary human monocytes. Importantly, the murine anti-hVISTA mAbs 7E12 and 7G5 displayed strong agonistic activity in human PBMC cultures; while Nb7 and rat anti-hmVISTA mAbs 3C3, 7C6, 7C7, and 7G1 also behaved as VISTA agonists, albeit to a lesser extent. Cross-reactive mAbs 7C7 and 7G1 further displayed agonistic potential in murine splenocyte assays. Importantly, mAb 7G1 significantly reduced inflammation associated with the murine model of Imiquimod-induced psoriasis. These agonistic VISTA mAbs may represent therapeutic leads to treat inflammatory disorders.

### Introduction

The immune system is regulated by a series of co-stimulatory and co-inhibitory signals. This delicate balance allows the immune system to protect against pathogens while preserving self-tolerance. Engagement of co-inhibitory [immune checkpoint] pathways involving PD-1:PD-L1 and CTLA-4:CD80/CD86 receptor-ligand pairs lead to T-cell suppression. Biologics that inhibit these immune checkpoint pathways have proven to be very effective in cancer immunotherapy,¹ while checkpoint agonists that activate these immune checkpoint pathways could be applied to treat autoimmune diseases or suppress inflammation.

V-domain immunoglobulin suppressor of T-cell activation (VISTA) is a recently discovered immune checkpoint that bears sequence homology to PD-L1. VISTA is expressed predominantly on the surface of hematopoietic cells, with the highest level of expression found on myeloid cells.^{2,3} Interestingly, VISTA has been proposed to function both as a ligand and as a receptor, as both exogenous VISTA²⁻⁶ and agonistic anti-VISTA antibodies⁷⁻⁹ suppress T-cell functions. Considerable effort has gone into generating antibodies that block the function of VISTA in the context of cancer immunotherapy. However, VISTA is an attractive therapeutic target for treating inflammatory disorders as well since VISTA-deficient mice display a higher basal level of immune activation¹⁰ and are more sensitive to developing ConA-induced hepatitis,⁸ encephalomyelitis,⁴ systemic lupus erythematosus,¹¹ asthma,¹² lupus erythematosus¹³ and exacerbates IMQ-induced psoriasiform inflammation of the ear.⁶

Here, we describe the generation of a new repertoire of agonistic anti-human VISTA (anti-hVISTA) monoclonal antibodies (mAbs) and nanobodies (Nbs) and demonstrate their functional activities. In particular, two human-specific mAbs 7E12 and 7G5 exhibited strong agonistic properties in suppressing ConA-induced activation of human T cells in whole PBMC cultures. In vivo use of cross-species anti-human/murine VISTA (anti-hmVISTA) mAb clone 7G1 in an Imiquimod (IMQ)-induced murine model of psoriasis exemplifies the therapeutic potential of agonizing VISTA for the treatment of inflammatory disorders.

### Materials and Methods

### Monoclonal Antibody Production

Hybridomas producing mAbs against recombinant human or murine VISTA (hVISTA or mVISTA) were generated by ImmunoPrecise Antibodies (Victoria, BC, Canada) by using a pentameric form comprising the extracellular IgV domain of human or murine VISTA (hVISTA-COMP or mVISTA-COMP)¹⁴ as the immunogen for vaccinating Balb/c mice or Lewis rats, respectively. Hybridomas were maintained in Dulbecco's modified Eagle medium (DMEM) (319-016-CL; WISENT) supplemented with 20% HyClone FetalClone II Serum (SH3010903; Cytiva), 1% penicillin-streptomycin (P/S) (450-200-EL; WISENT), 1×HT Supplement (11067030; Gibco), 1×2-mercaptoethanol (M6250; MilliporeSigma), and 25 mM HEPES (15630056; Gibco). Hybridomas were adapted to growth in H-CELL serum-free medium (001-035-CL; WISENT) for mAb purification. mAbs were purified from culture supernatants using HiTrap protein G HP columns (17040401; Cytiva) and eluted with 0.1M glycine-HCl (pH 2.7) neutralized with 1M Tris-HCl (pH 9.0). The recovered mAb preparations were buffer-exchanged into PBS using PD-10 columns (17085101; Cytiva), detoxified by passage through endotoxin removal columns (88274; Thermo Fisher Scientific) then either used immediately or stored at −20° C. The purity of mAbs was assessed by SDS-PAGE and Western blot using anti-mouse and anti-rat IgG heavy and light chain Ab conjugated to HRP (A110-105P; Bethyl Laboratories).

### Monoclonal Antibody Sequencing

mAbs were sequenced by RT-PCR followed by Sanger sequencing. Briefly, RNA was isolated from hybridomas using TRIzol Reagent (15596026; Thermo Fisher Scientific) and cDNA was synthesized using the SensiFAST cDNA synthesis kit (BIO-65053; Meridian Bioscience). Touch-down PCR was performed using DreamTaq DNA Polymerases (EP0701; Thermo Fisher Scientific) on a set of degenerate primers designed to sequence mouse¹⁵ and rat¹⁶ immunoglobulins (Integrated DNA Technologies). PCR amplification products were checked using agarose gel electrophoresis. PCR reactions that produced single products were treated with ExoSAP-IT PCR product cleanup reagent (78200.200.UL; Thermo Fisher Scientific) and sent in for Sanger sequencing at The Centre for Applied Genomics (SickKids hospital, Toronto, ON, Canada). Sequences were aligned against the IMGT database¹⁷ using IgBlast (https://www.ncbi.nlm.nih.gov/igblast/; National Center for Biotechnology Information, Bethesda, MD, USA)¹⁸ and grouped based on heavy chain CDR3 similarity using Clustal Omega (https://www.ebi.ac.uk/Tools/msa/clustalo/; EMBL-EBI, Hinxton, Cambridgeshire, UK).¹⁹

### Immunization, Library Construction, and Screening for Nanobodies

Nanobodies (Nbs) were generated as previously described.²⁰ Briefly, a llama (Lama glama) was immunized once weekly for 6 consecutive weeks with a preparation of hVISTA-COMP mixed with Gerbu LQ 3000 adjuvant (Gerbu Biotechnik GmbH). A Nb phage library was constructed following total RNA extraction and cDNA subcloning into the phagemid vector pMECS. Escherichia coli TG1 cells were transformed with this vector yielding 2×10⁸ transformants with 100% insert. Following 4 rounds of phage display panning using hVISTA-COMP, crude periplasmic extracts from 191 individual colonies were screened by ELISA for specificity towards hVISTA. Positive clones were selected for DNA sequencing (VIB sequencing facility, Flanders, Belgium). Sequence analysis was performed using CLC Main Workbench 8 software (Qiagen) and manually annotated in accordance to the IMGT numbering system.¹⁷ Selected anti-hVISTA Nbs were transformed into Escherichia coli WK6 to facilitate production.

### Nanobody Production

The expression and purification of Nbs followed protocols previously described.²¹ Briefly, 1 mL of an overnight starter culture of each of the clones was inoculated into 330 ml of Terrific Broth (TB) medium supplemented with 100 μg/ml ampicillin and 0.1% glucose and grown at 37° C. in shaking flasks (200 RPM) until OD600 nm reached 0.6-0.8. The cultures were subsequently induced by adding 1 mM isopropyl-β-D-thiogalactopyranoside (IPTG) and further incubated at 28° C. overnight (200 RPM). Cells were later harvested by centrifugation and incubated in 4 mL of TES (0.5 mM EDTA, 0.2 M Tris-HCL, 0.5 M sucrose, pH8.0) on ice, shaking (200 RPM) for 6 hours. An 8 mL aliquot of 0.25×TES (diluted in water) was then added to each cell suspension and the resulting mixtures incubated for an additional 12-15 hours before periplasmic extracts were collected via centrifugation. Nbs were purified from these extracts using immobilized metal affinity chromatography (IMAC) on a His-Trap column (17524802; Cytiva) and eluted with 0.5M Imidazole (1047160250; MilliporeSigma) in PBS. The eluted Nb fractions were desalted using PD-10 desalting columns (17085101; Cytiva) and detoxified using high-capacity endotoxin removal columns (88274; Thermo Fisher Scientific). The Nbs were either used immediately or stored at −20° C. The purity of each Nb was assessed by SDS-PAGE and Coomassie Blue staining. Western blot using a biotinylated anti-HA antibody (12158167001; MilliporeSigma) and streptavidin-HRP (RABHRP3; MilliporeSigma) was performed to confirm the presence of a Nb. A Nb (Nb19) targeting the babA adhesion molecule of H. pylori,²² was used as a negative control.

### Surface Plasma Resonance

Binding kinetics of mAbs and Nbs to hVISTA and mVISTA were obtained by surface plasmon resonance (SPR) using a BIAcore T200 (Cytiva) and HBS running buffer (20 mM of HEPES pH 7.4, 150 mM NaCl, 0.005% Tween-20, 3.4 mM EDTA). Briefly, anti-histidine antibodies were immobilized on a CM5 chip (29149604; Cytiva) using the His capture kit (28995056; Cytiva) following manufacture's protocol. For mAbs, VISTA was captured by flowing 30 mg/ml of hVISTA-COMP-his or mVISTA-COMP-his at a flow rate of 30 μL/min. Sensorgrams were collected using single-cycle kinetics covering five mAb concentrations (1:2 serial dilutions). For Nbs, solutions of each nanobody (30 mg/mL) were flown over an anti-histidine-coated chip. Five concentrations (1:2 serial dilutions) of hVISTA-Fc (produced in house) were then injected at a flow rate of 30 μL/min. K_{D}, Kₒₙ, and K_{off} were calculated by analyzing the sensorgrams with a 1:1 Langmuir binding model²³ after subtracting reference sensorgrams.

### Competition ELISA

To confirm the specificity of the Nbs to hVISTA, a competition ELISA was performed against anti-hVISTA mAbs. ELISA plate wells (44-2404-21; Thermo Fisher Scientific) were coated with hVISTA-COMP (10 μg/ml) and subsequently blocked with 0.5% milk. Nbs (330 nM) were then dispensed into wells alone or in combination with 2-fold molar excess of mAbs, mouse IgG1 control (400165; BioLegend), or rat IgG2a control (400543; BioLegend), and incubated for 1 hour at room temperature. hVISTA:Nb complexes were detected using a biotinylated anti-HA antibody (12158167001; MilliporeSigma), followed by streptavidin HRP (RABHRP3; MilliporeSigma) and the substrate TMB (3,3′,5,5′-tetramethylbenzidine) (34028; Thermo Fisher Scientific). Absorbance readings were recorded at 450 nm.

### Mice

Female C57BL/6 mice at 8-10 weeks of age (The Jackson Laboratory) were used throughout this study and housed at the Sunnybrook Research Institute (SRI; Sunnybrook Health Sciences Centre, Toronto, ON, Canada) Comparative Research (SRICR) facility. All protocols were approved by the SRICR Animal Care Committee, accredited by the Canadian Council of Animal Care.

### Direct Cell Binding Assay

Binding of mAbs and Nbs to human and murine immune cells were tested using human PBMCs and mouse splenocytes. For binding on human cells, whole human blood was obtained from donors in accordance with guidelines set forth by the Sunnybrook Research Ethics board (REB approval number 443-2017). Human peripheral blood mononuclear cells (PMBCs) were isolated from whole blood using Ficoll Paque PLUS (1.077 g/ml) (17144003; Cytiva). PBMCs had their Fc receptors blocked with Human TruStain FcX (422301; BioLegend) then incubated with 2.5 μg of mAb, mouse IgG1 (401408; BioLegend), or rat IgG2a (400565; BioLegend) or 15 μg Nb for 1 hour at 4° C. After washing with PBS, the PBMCs were incubated with secondary antibodies (1 μL; FITC anti-mouse IgG1, FITC anti-HA, or FITC anti-rat IgG2a; 406605, 901507, or MRG2a-83; BioLegend) or control FITC anti-VISTA antibodies (clone B7H5DS8; 11-1088-42; eBioscience) for 40 minutes at 4° C. A staining cocktail consisting of PE/Cy5 anti-CD11b (101210; BioLegend), APC/Cy7 anti-CD14 (325620; BioLegend), Alexa Flour 700 anti-CD16 (302026; BioLegend), PE/Cy7 anti-CD4 (317414; BioLegend), and Alexa Flour 647 anti-CD8 (301022; BioLegend) was then added to the PBMCs and incubated for 20 minutes at 4° C. Finally, the PBMCs were washed and resuspended in 3 μM DAPI (D1306; Thermo Fisher Scientific) and cytometric profiles recorded using a BD LSR II flow cytometer (BD) maintained by The Centre for Flow Cytometry & Scanning Microscopy (CCSM) at Sunnybrook Research Institute (SRI; Sunnybrook Health Sciences Centre, Toronto, ON, Canada). The same protocol was performed on murine splenocytes to measure the binding of rat anti-hmVISTA mAbs to murine immune cells. The staining cocktail consisted of APC/Cy7 anti-CD45 (147718; BioLegend), BV510 anti-CD11b (101263; BioLegend), Alexa Flour 700 anti-CD3 (100216; BioLegend), Alexa Flour 647 anti-CD8 (100724; BioLegend), PE/Dazzle 594 anti-Ly6C (128044; BioLegend), PE/Cy7 anti-Ly6G (127618; BioLegend), FITC anti-F4/80 (123108; BioLegend), Pacific Blue anti-MHCII (107620; BioLegend), and PerCP/Cy5.5 anti-CD11c (117328; BioLegend).

### T Cell Proliferation and Cytokine Assays on Human PBMCs

Human PBMCs were stained with 5 UM CFSE (C34554; Thermo Fisher Scientific) and cultured in X-VIVO 15 medium (BE02-060F; Lonza) supplemented with 5% fetal bovine serum (FBS; 080-450; WISENT) in round-bottom 96-well plates at 30,000 cells per well. ConA (1 μg/mL; C5275; MilliporeSigma) was used to stimulate the PBMCs and 15 μg/mL of either mAbs, mouse IgG1 controls (400165; BioLegend), rat IgG2a controls (400544; BioLegend), or Nbs were added and topped up to 300 μL per well. Supernatants were collected on day 2 for cytokine analyses using LegendPlex kits for IL-2, IFNγ, and IL-10 (BioLegend). Cells were collected after 4 days of culture, stained as described above, and analysed by flow cytometry to measure T cell proliferation.

### Mouse Splenocyte Proliferation Assay

Mouse splenocytes were treated with red blood cell lysis buffer, stained with 5 UM CFSE (C34554; Thermo Fisher Scientific), and cultured in X-VIVO 15 medium (BE02-060F; Lonza) supplemented with 5% FBS (080-450; WISENT) in round-bottom 96-well plates at 30,000 cells per well. ConA (2 μg/mL; C5275; MilliporeSigma) was used to stimulate the PBMCs and 15 μg/mL of each mAb or a rat IgG2a control (400544; BioLegend) were added and topped up to 300 μL per well. Cells were collected after 4 days of culture, stained with a cocktail consisting of APC/Cy7 anti-CD45 (147718; BioLegend), Alexa Flour 700 anti-CD3 (100216; BioLegend), Alexa Flour 647 anti-CD8 (100724; BioLegend), and PE/Cy7 anti-CD4 (100422; BioLegend), and analysed by flow cytometry.

### IMQ-Induced Psoriasis in Mice

Ten-week old female C57BL/6 mice were treated with a daily dose of 62.5 mg of imiquimod (IMQ) cream (Zyclra; 3.75% w/v) or petroleum jelly (Vaseline) control applied evenly on their shaven back with a cotton swab. Daily doses (100 μg) of either an anti hmVISTA mAb, or a rat IgG2a control (400544; BioLegend), or PBS were injected intraperitoneally into control or IMQ-treated mice. Mice were monitored daily for severity of the psoriasis-like skin conditions: erythema, scaling, and thickness.²⁴ Each of the conditions were scored from 0 to 4, with 4 being very marked, by 3 independent scorers who were blinded to the treatment type.

Mice were sacrificed on days 3 and 6 and had their skin harvested for qPCR analysis to profile for gene expression. Briefly, skin areas that were shaven and treated with IMQ or Vaseline cream were excised and chopped finely before being treated with TRIzol Reagent (15596026; Thermo Fisher Scientific) to isolate RNA contents, which were then reverse-transcribed using high-capacity cDNA reverse transcription kit (4368814; Thermo Fisher Scientific). qPCR was performed with the SensiFAST SYBR no-ROX kit (BIO-98005; Meridian Bioscience) and gene-specific primers listed in Table 1 (Integrated DNA Technologies) and read on the Mastercycler ep realplex qPCR instrument (Eppendorf). In addition, mice sacrificed on day 6 had their splenocytes harvested and profiled for CD4⁺ and CD8⁺ T cells with a staining cocktail containing APC/Cy7 anti-CD45 (147718; BioLegend), Alexa Flour 700 anti-CD3 (100216; BioLegend), Alexa Flour 647 anti-CD8 (100724; BioLegend), and PE/Cy7 anti-CD4 (100422; BioLegend).

### Statistics

Each PBMC experiment was repeated on 5 different donors that were randomized between groups. Student's t-test (2-tail, paired, adjusted for multiple comparisons where necessary) was applied to test significance between PBMCs treated with ConA alone or ConA in the presence of mAbs or Nbs (significance was taken at α=0.05).

### Results

Characterization of Anti-VISTA mAbs and Nbs

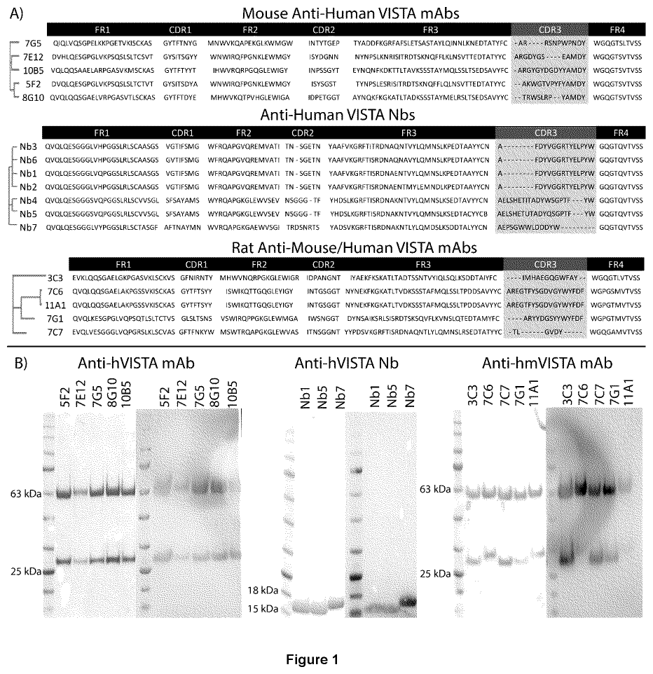

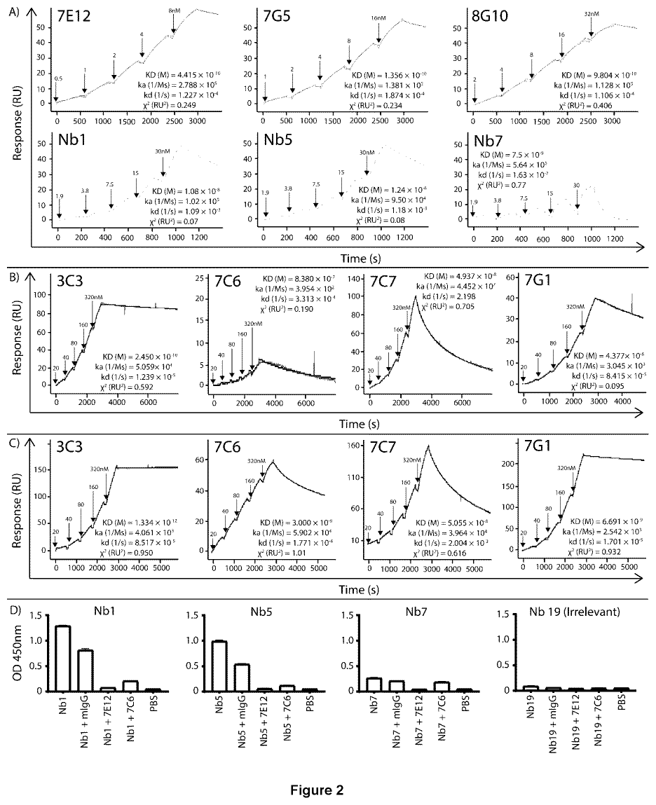

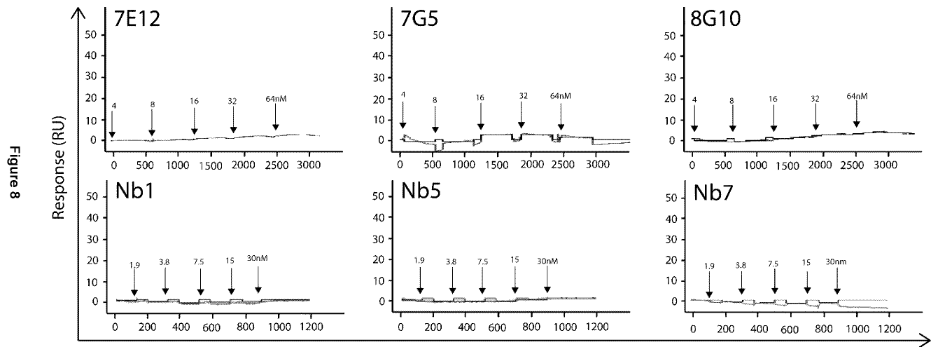

Following ELISA screenings against human and mouse VISTA (hVISTA and mVISTA), five hybridoma clones generating murine anti-hVISTA mAbs (7G5, 7E12, 10B5, 5F2 and 8G10) and five hybridoma clones generating rat anti-hmVISTA mAbs (3C3, 7C6, 7C7, 7G1, and 11A1) were selected and further expanded based on their production levels. All of the selected murine clones generated mouse IgG1 anti-hVISTA mAbs. The variable regions of the five chosen clones were sequenced and pooled into three different families based on similarities in their heavy chain CDR3 (FIG. 1A), a region typically associated with mAb specificity.²⁵ The presence of heavy and light chains as well as the purity of mAbs were confirmed by SDS-PAGE and by Western blot detected using an anti-mouse IgG heavy and light chain antibody (FIG. 1B). Representative mAbs from each of the three families, namely 7E12, 7G5, and 8G10, were chosen for further characterization based on their sub-nanomolar binding (equilibrium dissociation constant; K_{D}) to human VISTA (K_{D} values of 0.44 nM, 0.14 nM, and 0.98 nM, respectively) as measured by surface plasmon resonance (SPR) (FIG. 2A). As expected, none of these murine antibodies recognized recombinant mouse VISTA (FIG. 8). In addition to the murine anti-hVISTA clones, five rat hybridoma clones were selected based on their production of mAbs that bind to both human and murine VISTA. All five clones produce mAbs (FIG. 1B) of the rat IgG2a subtype. As sequencing shows that two of the rat mAbs (7C6 and 11A1) have the same heavy chain sequence, four hybridoma clones were selected for further characterization (3C3, 7C6, 7C7, and 7G1) (FIG. 1A). All of the selected clones bound to both hVISTA (K_{D} values of 0.24 nM, 840 nM, 49 nM, and 44 nM, respectively; FIG. 2B) and mVISTA (K_{D} values of 1.3 μM, 3 nM, 51 nM, and 6.7 nM, respectively; FIG. 2C).

In addition to the mAbs, seven positive anti-hVISTA Nbs phage clones (hVISTANb1 to 7; Nb1 to Nb7) were identified from bio-panning against hVISTA. The seven anti-hVISTA Nbs phage clones identified were regrouped into three families based on amino acid sequence differences within their CDR domains (FIG. 1A). One clone corresponding to each of the families, termed Nb1, Nb5, and Nb7, was chosen for production and characterization. All three Nbs were successfully purified as evidenced by the expected molecular weight band (˜15 kDa) on SDS-PAGE and by Western blot (FIG. 1B). Nb1, Nb5, and Nb7 bound to hVISTA with K_{D} in the low nM range (11 nM, 12 nM, and 7.5 nM, respectively) as determined by SPR (FIG. 2A). None of the Nbs recognized mVISTA (FIG. 8).

To further confirm the selectivity and specificity of the Nbs for hVISTA, a competition ELISA was performed to assess the ability of selected mAbs to displace the binding of Nbs to hVISTA. FIG. 2D shows that 2-fold molar excess of the representative anti-hVISTA mAb 7E12 but not the control mouse IgG1, was able to fully displace Nb1, Nb5, and Nb7 from binding to hVISTA. The irrelevant control Nb19 did not bind hVISTA. Notably, a representative of the cross-reactive anti-hmVISTA family, mAb 7C6, only partially displaced the Nbs from binding to hVISTA, suggesting that the cross-reactive mAb binds a partly different epitope from the mAbs that bind specifically to human VISTA.

mAbs Optimally Detect VISTA on Primary Human and Murine Myeloid Cells

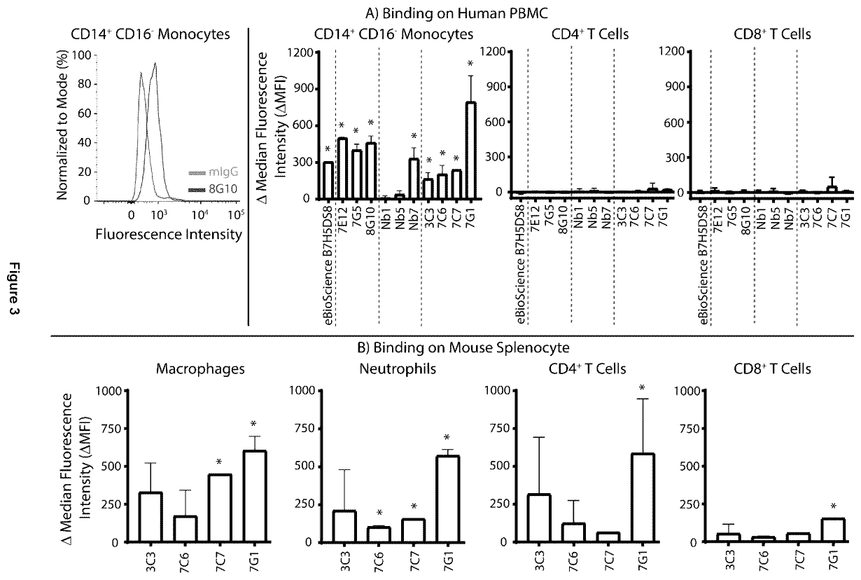

VISTA is predominately expressed on cells of the myeloid compartment (monocytes, macrophages, neutrophils, and dendritic cells) and to a lesser extent in naïve CD4⁺ and CD8⁺ T cells.^{3,4,8} Flow cytometry was thus used to detect the binding of selected mAbs and Nbs to CD14⁺CD16⁻ classical monocytes, CD4⁺ T cells, and CD8⁺ T cells in PBMCs isolated from healthy donors. As shown in FIG. 3A, all of the mAbs investigated (anti-hVISTA mAbs 7E12, 7G5, and 8G10 and anti-hmVISTA mAbs 3C3, 7C6, 7C7, and 7G1) detected VISTA expression on human classical monocytes, at a comparable level as the commercially available positive control mAb (clone B7H5DS8; eBioscience). No binding was detected on either T cell subset. However, out of the anti-hVISTA Nbs, only Nb7 was able to strongly detect VISTA expression on human classical monocytes. As was the case for all mAbs, none of the Nbs detected VISTA expression on T cells. As such, the functional characterization of only the 3 murine anti-human VISTA mAbs and Nb7 were subsequently pursued.

Similarly, the cross-species anti-hmVISTA mAbs were tested for their ability to bind immune cell populations in mouse splenocytes. As shown in FIG. 3B, all of the mAbs bind mouse macrophages and neutrophils at a comparable level to the mAbs' binding on human CD14⁺CD16⁻ monocytes, although binding for mAb 3C3 was not statistically significant. As opposed to binding on human PBMCs, the mAbs also detected VISTA expression on CD4⁺ T cells, which is in accordance with previous studies.³ Notably, out of all mAbs tested, 7G1 exhibited the highest binding to both human and murine immune cell populations known to express VISTA.

Anti-hVISTA mAbs 7E12 and 7G5 Display Strong Agonistic Potential

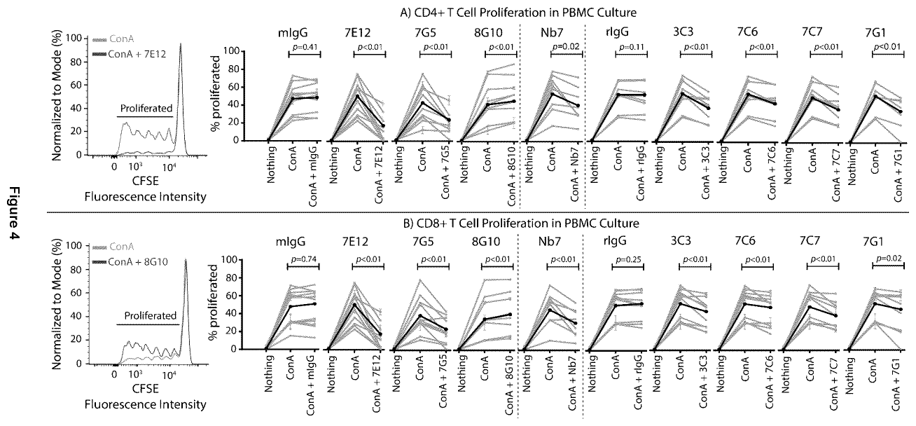

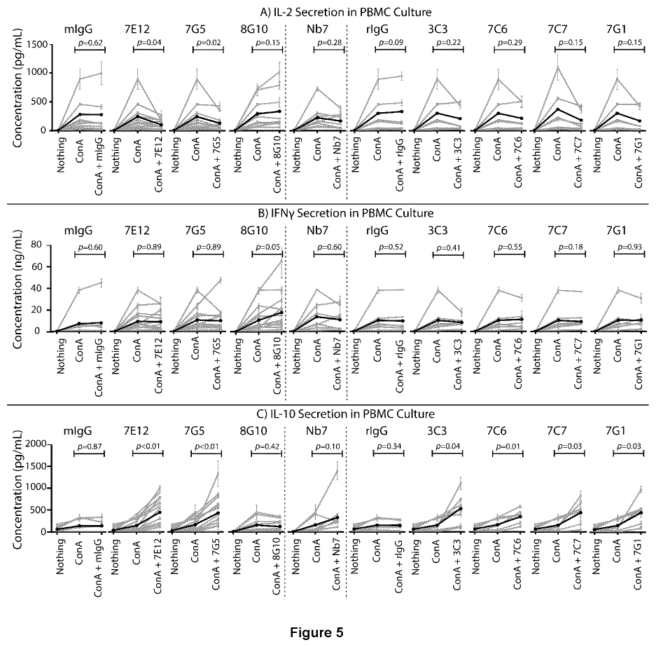

From a functional standpoint, VISTA is described as a negative checkpoint regulator of immune responses. Accordingly, agonizing or antagonizing VISTA is potentially consequential for the treatment of inflammatory conditions or cancer, respectably. Ex vivo experiments were thus conducted with human PBMCs to define if anti-hVISTA mAbs 7E12, 7G5, and 8G10, anti-hVISTA Nb7, and anti-hmVISTA mAbs 3C3, 7C6, 7C7, and 7G1 behave as human VISTA agonists or antagonists. Herein, human PBMC cultures were stimulated with concanavalin A (ConA; 1 μg/mL) in the presence or absence of mAbs or Nb7. CD4⁺ and CD8⁺ T-cell proliferation responses (FIG. 4) and cytokine levels (FIG. 5) were subsequently assessed.

Two of the mAbs, 7E12 and 7G5, strongly inhibited the proliferation of CD4⁺ and CD8⁺ T cells induced by ConA (FIG. 4), caused a reduction in ConA-induced pro-inflammatory cytokine IL-2 production, and led to a significant increase in levels of the anti-inflammatory cytokine IL-10 when compared to ConA alone (FIG. 5), suggesting that both these mAbs behave as hVISTA agonists. In particular, 7E12 displayed the greatest reduction in both CD4⁺ and CD8⁺ T-cell proliferation (65% and 62%, respectively) and may be an ideal candidate for treating human inflammation-associated disease processes.

In contrast, mAb 8G10 acts as a weak human VISTA antagonist, mildly enhancing the proliferation of both CD4⁺ and CD8⁺ T cells as compared to ConA activation alone (p<0.01; FIG. 4). Moreover, IFNγ levels were statistically elevated in human PBMC cultures treated with ConA and 8G10 compared to those treated with ConA alone (p<0.05; FIG. 5).

As shown in FIG. 4, Nb7 behaved as a human VISTA agonist based on T-cell proliferation, whereby the proliferation of both CD4⁺ and CD8⁺ T cells were significantly suppressed when compared to ConA activation alone. However, as shown in FIG. 5, levels of IL-2, IFNγ, and IL-10 in the human PMBC groups treated with ConA and Nb7 were not statistically different than the levels observed for those treated with ConA alone.

All of the cross-reactive anti-hmVISTA mAbs (3C3, 7C6, 7C7, and 7G1) appear to behave agonistically in the ConA-stimulated human PBMC cultures, reducing the proliferation of CD4⁺ and CD8⁺ T cells, although to a weaker extent than the mAbs that target human VISTA specifically (FIG. 4). None of the anti-hmVISTA mAbs affected the secretion levels of IL-2 and IFNγ significantly, but all of them significantly increased the production of the anti-inflammatory cytokine IL-10 (FIG. 5).

### Anti-hmVISTA mAb 7G1 Reduced the Severity of Psoriasis-Like Symptoms in Mice

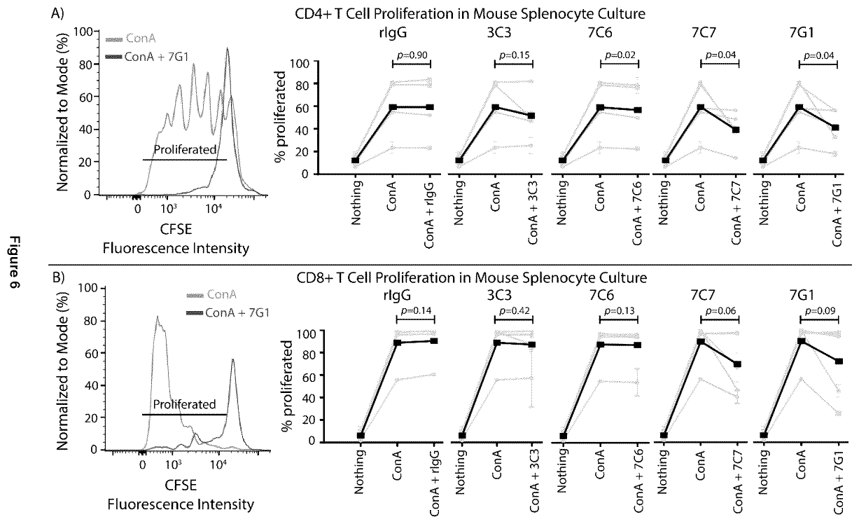

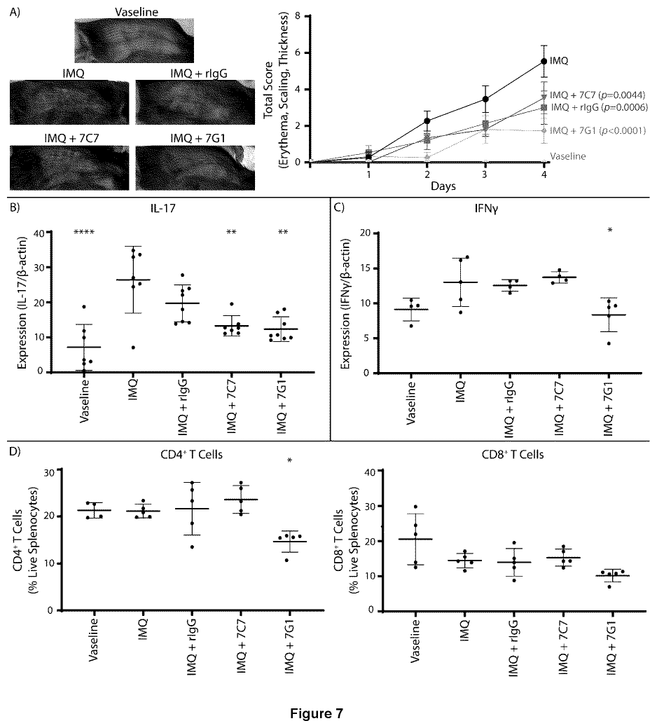

Before testing the cross-species anti-hmVISTA mAbs in vivo, the mAbs were first screened in an ex vivo experiment on ConA-stimulated mouse splenocytes in a similar set-up as described above for human PBMCs. As shown in FIGS. 6, 7C6, 7C7, and 7G1 reduced the proliferation of ConA-stimulated CD4⁺ T cells significantly (4%, 34%, and 31%, respectively), although the reduction was weak for 7C6. In addition, mouse splenocytes treated with ConA and either 7C7 or 7G1 also displayed lower proliferation rates of CD8⁺ T cells as compared to those treated with ConA alone (22% and 20%, respectively), although the differences were not statistically significant. Therefore, 7C7 and 7G1 were chosen to be further tested in vivo.

Agonistic anti-hmVISTA mAbs were tested to treat inflammatory responses in a IMQ-induced psoriasis-like model in mice²⁴ as VISTA deficiency was previously shown to exacerbate a form of IMQ-induced psoriasis inflammation of the ear.⁶ In this model, IMQ cream (62.5 mg) was applied topically on a daily basis to a shaved area of female C57BL/6 mice, where IMQ activates immune responses as a TLR7 and TLR8 ligand. IMQ-treated mice received 100 μg of an mAb intraperitoneally every other day. As FIG. 7A shows, 7G1 significantly reduced the severity score of IMQ-induced psoriasis-like skin inflammation. Interestingly, although 7C7 also ameliorated the symptoms of skin inflammation, the degree of change was less than that of rat IgG2a control. Since IL-17 and TH17 cells play a pivotal role in psoriasis, IL-17 expression level in the skin was measured by qPCR. As demonstrated in FIG. 7B, IL-17 expression was significantly increased after IMQ stimulation, and was significantly reduced by both 7C7 and 7G1 (by 50% and 53%, respectively), and not by rat IgG2a control. Meanwhile, the expression of pro-inflammatory cytokine IFNγ was significantly lowered by 7G1 (by 36%) only (FIG. 7C). Finally, since the depletion of T cells had been shown to attenuate psoriasis-like symptoms,²⁴ the effect of mAbs on T cell populations in the spleen was measured using flow cytometry. Indeed, mice treated with 7G1 displayed a significant reduction in the percentage of CD4⁺ T cells in the spleen (FIG. 7D), although the change in CD8⁺ T cells was not significant. Overall, 7G1 is a strong agonist of human and murine VISTA that not only reduces the proliferation of T cells and pro-inflammatory cytokine secretion ex vivo, but also attenuates psoriasis-like inflammation in vivo.

### Discussion

VISTA is part of a co-inhibitory immune pathway where it functions as both a ligand and a receptor. Since the VISTA pathway is functionally distinct from the PD-1:PD-L1 axis, it suggests that VISTA antagonists may be paired with approved immune checkpoint inhibitors in cancer immunotherapy. As important, VISTA agonists may be valuable in treating inflammation and autoimmunity. Herein, we describe the generation and functional characterization of murine mAbs and heavy chain single domain antibody fragments (Nbs) that specifically bind to the human VISTA extracellular domain, as well as rat mAbs that bind to both human and murine VISTA. Importantly, the study identified two strongly agonistic anti-hVISTA mAbs (7E12 and 7G5) that suppress T cell proliferation in ConA-stimulated human PBMC cultures; and an agonistic anti-hmVISTA mAb (7G1) that attenuated IMQ-induced psoriasis in vivo.

All VISTA binders bound with nanomolar (nM) affinities to human or human/murine VISTA (FIG. 2). In contrast, studies have shown that monomeric forms of IgV domains, PD1/PD-L1, for example, interact with each other in the low micromolar (μM) range.²⁶ These anti-VISTA mAbs and Nbs detected VISTA in the context of both ELISA and flow cytometry experiments. Interestingly, Nb7 displayed the fastest dissociation constant upon binding to hVISTA, yet generated the strongest mean fluorescence intensity signal of all 3 Nbs in detecting VISTA-expressing CD14⁺CD16⁻ human monocytes by flow cytometry (FIG. 3).

Since a previously reported agonistic mAb against murine VISTA was able to protect mice from ConA-induced hepatitis,⁸ we tested our anti-human VISTA biologics for their therapeutic potential by studying their effect on a ConA-stimulated human PBMC culture. ConA is a lectin that has been shown to activate T cells and induce T cell-mediated tissue inflammation in mice.²⁷ In a PBMC culture, monocytes are capable of presenting VISTA, as shown by previous studies^{3,4,8} and confirmed by our cell binding assay using flow cytometry (FIG. 3), to T cells. As such, PBMC cultures better simulate the in vivo environment as opposed to using purified T cells with exogenous VISTA. Here, mAb 8G10 increased the proliferation of CD4⁺ and CD8⁺ T cells slightly (FIG. 4) and enhanced the production of IFNγ in ConA-stimulated human PBMC cultures. This result suggests that 8G10 may act as a hVISTA antagonist, which may reflect its ability to block the interaction between human VISTA on monocytes and VISTA receptors on T cells. In contrast, all other mAbs and Nb7 reduced the proliferation of human T cells (FIG. 4). However, based on minimal changes in expression patterns observed for both pro and anti-inflammatory cytokines relative to the ConA activation of whole human PBMCs from multiple donors, Nb7 does not represent a strong human VISTA agonist (FIG. 5).

Treatment of ConA-activated human PBMCs with anti-hVISTA mAbs 7E12 and 7G5 strongly inhibited CD4⁺ and CD8⁺ T cell proliferation and reduced the production of IL-2, a T cell-activating cytokine,²⁸ while increasing the expression of IL-10, which inhibits T cell proliferation and IL-2 production,²⁹ confirming them as human VISTA agonists (FIGS. 4 and 5). The agonistic potential of these mAbs may be mediated not only through the direct suppression of VISTA on T cells,⁸ but also indirectly via the high expression of VISTA in the myeloid compartment, where suppressed monocytes downregulate T cell activation.

Similarly, the agonistic property of anti-hmVISTA mAbs were tested using mouse splenocytes and in an in vivo mouse model of IMQ-induced psoriasis. All of the anti-hmVISTA mAbs detected VISTA expression on murine macrophages, neutrophils, and CD4⁺ cells, with 7G1 being the strongest binder to these VISTA-expressing immune cell populations (FIG. 3B). However, only 7C7 and 7G1 reduced the proliferation of CD4⁺ T cells in ConA-stimulated mouse splenocyte cultures (FIG. 6); and only 7G1 was able to alleviate the severity of IMQ-induced psoriasis-like skin inflammation beyond rat IgG control (FIG. 7A). The most surprising result in this model was the ability of the rat IgG2a control to attenuate IMQ-induced inflammation. This finding may be due to the sialylation of this IgG as shown by the anti-inflammatory properties of purified IgG fractions given intravenously during gamma globulin therapy.³⁰ The potential of mAb 7G1 to reduce the severity of psoriasis-like symptoms may be linked to its ability to reduce splenic T cells (FIG. 7D) and the epidermal expression of IL-17 (FIG. 7B), a critical cytokine in the development of plaque-like psoriasis.²⁴ Furthermore, IFNγ expression is reduced by mAb 7G1 (FIG. 7C). IFNγ represents a prognostic marker of psoriasis where a decrease in IFNγ predicts a lower score in terms of psoriasis severity³¹ although data on the level of IFNγ observed between psoriatic lesions and healthy controls remain contradictory.³² This observation may explain why no difference was found between IFNγ levels in Vaseline- and IMQ-treated mice.

Although this study did not identify anti-VISTA Nbs as useful agonists or antagonists, it is believed that these could be designed and tested successfully. Alterative selection criteria, stringency in the method, or alternative immunization strategies may yield more favorable binders, potentially with antagonistic and/or cross-reactive properties. Furthermore, alternative Nb engineering strategies including their multimerization to create higher avidity constructs, may improve their clinical usefulness. Bivalency for example, by fusing a Nb to an Fc domain may improve upon the therapeutic potential of all Nbs described in this study.

In conclusion, we have generated a panel of mAbs and Nbs targeting human VISTA and demonstrated their utility in detecting the presence of human VISTA in ELISA and flow cytometry assays. Importantly, we demonstrated the agonistic properties of anti-hVISTA mAbs 7E12 and 7G5 on human PBMCs in the context of an inflammation-driven microenvironment (ConA stimulation) and the ability of agonist anti-hmVISTA mAb 7G1 in attenuating IMQ-induced psoriasis-like skin inflammation. These findings suggest that VISTA agonists, such as agonistic anti-VISTA mAbs, will prove useful in treating inflammatory or auto-immune diseases.

### REFERENCES

1. Pardoll D M. The blockade of immune checkpoints in cancer immunotherapy. Nat Rev Cancer 2012; 12:252-64.
2. Flies D B, Wang S, Xu H, Chen L. Cutting Edge: A Monoclonal Antibody Specific for the Programmed Death-1 Homolog Prevents Graft-versus-Host Disease in Mouse Models. J Immunol 2011; 187:1537-41.
3. Wang L, Rubinstein R, Lines J L, Wasiuk A, Ahonen C, Guo Y, Lu L-FF, Gondek D, Wang Y, Fava R A, et al. VISTA, a novel mouse Ig superfamily ligand that negatively regulates T cell responses. J Exp Med 2011; 208:577-92.
4. Lines J L, Pantazi E, Mak J, Sempere L F, Wang L, O'Connell S, Ceeraz S, Suriawinata A A, Yan S, Ernstoff M S, et al. VISTA is an immune checkpoint molecule for human T cells. Cancer Res 2014; 74:1924-32.
5. Le Mercier I, Chen W, Lines J L, Day M, Li J, Sergent P, Noelle R J, Wang L. VISTA regulates the development of protective antitumor immunity. Cancer Res 2014; 74:1933-44.
6. Li N, Xu W, Yuan Y, Ayithan N, Imai Y, Wu X, Miller H, Olson M, Feng Y, Huang Y H, et al. Immune-checkpoint protein VISTA critically regulates the IL-23/IL-17 inflammatory axis. Sci Rep 2017; 7:1485.
7. Flies D B, Higuchi T, Chen L. Mechanistic Assessment of PD-1H Coinhibitory Receptor-Induced T Cell Tolerance to Allogeneic Antigens. J Immunol 2015; 194:5294-304.
8. Flies D B, Han X, Higuchi T, Zheng L, Sun J, Ye J J, Chen L. Coinhibitory receptor PD-1H preferentially suppresses CD4+ T cell-mediated immunity. J Clin Invest 2014; 124: 1966-75.
9. ElTanbouly M A, Zhao Y, Nowak E, Li J, Schaafsma E, Le Mercier I, Ceeraz S, Lines J L, Peng C, Carriere C, et al. VISTA is a checkpoint regulator for naïve T cell quiescence and peripheral tolerance. Science (80-) 2020; 367:eaay0524.
10. Yoon K W, Byun S, Kwon E, Hwang S Y S-Y, Chu K, Hiraki M, Jo S H S-H, Weins A, Hakroush S, Cebulla A, et al. Control of signaling-mediated clearance of apoptotic cells by the tumor suppressor p53. Science (80-) 2015; 349:1-34.
11. Ceeraz S, Sergent P A, Plummer S F, Schned A R, Pechenick D, Burns C M, Noelle R J. VISTA Deficiency Accelerates the Development of Fatal Murine Lupus Nephritis. Arthritis Rheumatol 2017; 69:814-25.
12. Liu H, Li X, Hu L, Zhu M, He B, Luo L, Chen L. A crucial role of the PD-1H coinhibitory receptor in suppressing experimental asthma. Cell Mol Immunol 2018; 15:838-45.
13. Han X, Vesely M D, Yang W, Sanmamed M F, Badri T, Alawa J, López-Giráldez F, Gaule P, Lee S W, Zhang J-P P, et al. PD-1H (VISTA)-mediated suppression of autoimmunity in systemic and cutaneous lupus erythematosus. Sci Transl Med 2019; 11:1-15.
14. Prodeus A, Abdul-Wahid A, Sparkes A, Fischer N W, Cydzik M, Chiang N, Alwash M, Ferzoco A, Vacaresse N, Julius M, et al. VISTA.COMP—an engineered checkpoint receptor agonist that potently suppresses T cell-mediated immune responses. JCI Insight 2017; 2.
15. Chardès T, Villard S, Ferrières G, Piechaczyk M, Cerutti M, Devauchelle G, Pau B. Efficient amplification and direct sequencing of mouse variable regions from any immunoglobulin gene family. FEBS Lett 1999; 452:386-94.
16. Sepulveda J, Shoemaker C B. Design and testing of PCR primers for the construction of scFv libraries representing the immunoglobulin repertoire of rats. J Immunol Methods 2008; 332:92-102.
17. Lefranc M-P, Pommié C, Ruiz M, Giudicelli V, Foulquier E, Truong L, Thouvenin-Contet V, Lefranc G. IMGT unique numbering for immunoglobulin and T cell receptor variable domains and Ig superfamily V-like domains. Dev Comp Immunol 2003; 27:55-77.
18. Ye J, Ma N, Madden T L, Ostell J M. IgBLAST: an immunoglobulin variable domain sequence analysis tool. Nucleic Acids Res 2013; 41:W34-40.
19. Sievers F, Wilm A, Dineen D, Gibson T J, Karplus K, Li W, Lopez R, McWilliam H, Remmert M, Söding J, et al. Fast, scalable generation of high-quality protein multiple sequence alignments using Clustal Omega. Mol Syst Biol 2011; 7:539.
20. C, Vincke, Gutiérrez C, Wernery U, Devoogdt N, Hassanzadeh-Ghassabeh G M S. Generation of single domain antibody fragments derived from camelids and generation of manifold constructs. Methods Mol Biol 2012; 907:145-76.
21. Conrath K E, Lauwereys M, Galleni M, Matagne A, Frère J M, Kinne J, Wyns L, Muyldermans S. β-Lactamase inhibitors derived from single-domain antibody fragments elicited in the Camelidae. Antimicrob Agents Chemother 2001; 45:2807-12.
22. Romão E, Krasniqi A, Maes L, Vandenbrande C, Sterckx Y G-J, Stijlemans B, Vincke C, Devoogdt N, Muyldermans S. Identification of Nanobodies against the Acute Myeloid Leukemia Marker CD33. Int J Mol Sci 2020; 21:310.
23. Karlsson R, Katsamba P S, Nordin H, Pol E, Myszka D G. Analyzing a kinetic titration series using affinity biosensors. Anal Biochem 2006; 349:136-47.
24. van der Fits L, Mourits S, Voerman J S A, Kant M, Boon L, Laman J D, Cornelissen F, Mus A, Florencia E, Prens E P, et al. Imiquimod-Induced Psoriasis-Like Skin Inflammation in Mice Is Mediated via the IL-23/IL-17 Axis. J Immunol 2009; 182:5836-45.
25. Xu J L, Davis M M. Diversity in the CDR3 Region of VH Is Sufficient for Most Antibody Specificities. Immunity 2000; 13:37-45.
26. Lin D Y-w., Tanaka Y, Iwasaki M, Gittis A G, Su H-P, Mikami B, Okazaki T, Honjo T, Minato N, Garboczi D N. The PD-1/PD-L1 complex resembles the antigen-binding Fv domains of antibodies and T cell receptors. Proc Natl Acad Sci 2008; 105:3011-6.
27. Tiegs G, Hentschel J, Wendel A. A T cell-dependent experimental liver injury in mice inducible by concanavalin A. J Clin Invest 1992; 90:196-203.
28. Taniguchi T, Minami Y. The IL-2/IL-2 receptor system: A current overview. Cell 1993; 73:5-8.
29. Taga K, Tosato G. IL-10 inhibits human T cell proliferation and IL-2 production. J Immunol 1992; 148:1143-8.
30. Kaneko Y, Nimmerjahn F, Ravetch J V. Anti-inflammatory activity of immunoglobulin G resulting from Fc sialylation. Science (80-) 2006; 313:670-3.
31. Abdallah M A, Abdel-Hamid M F, Kotb A M, Mabrouk E A. Serum interferon-γ is a psoriasis severity and prognostic marker. Cutis 2009; 84:163-8.
32. Pietrzak A T, Zalewska A, Chodorowska G, Krasowska D, Michalak-Stoma A, Nockowski P, Osemlak P, Paszkowski T, Roliński J M. Cytokines and anticytokines in psoriasis. Clin Chim Acta 2008; 394:7-21.
33. Deng J, Li J, Sarde A, Lines J L, Lee Y-CC, Qian D C, Pechenick D A, Manivanh R, Le Mercier I, Lowrey C H, et al. Hypoxia-induced VISTA promotes the suppressive function of myeloid-derived suppressor cells in the tumor microenvironment. Cancer Immunol Res 2019; 7:1079-90.
34. Wang L, Jia B, Claxton D F, Ehmann W C, Rybka W B, Mineishi S, Naik S, Khawaja M R, Sivik J, Han J, et al. VISTA is highly expressed on MDSCs and mediates an inhibition of T cell response in patients with AML. Oncoimmunology 2018; 7:e1469594.
35. Blando J, Sharma A, Higa M G, Zhao H, Vence L, Yadav S S, Kim J, Sepulveda A M, Sharp M, Maitra A, et al. Comparison of immune infiltrates in melanoma and pancreatic cancer highlights VISTA as a potential target in pancreatic cancer. Proc Natl Acad Sci 2019; 116:1692-7.
36. Gao J, Ward J F, Pettaway C A, Shi L Z, Subudhi S K, Vence L M, Zhao H, Chen J, Chen H, Efstathiou E, et al. VISTA is an inhibitory immune checkpoint that is increased after ipilimumab therapy in patients with prostate cancer. Nat Med 2017; 23:551-5.

The above disclosure generally describes the present invention. Although specific terms have been employed herein, such terms are intended in a descriptive sense and not for purposes of limitation.

All publications, patents and patent applications cited above are herein incorporated by reference in their entirety to the same extent as if each individual publication, patent or patent application was specifically and individually indicated to be incorporated by reference in its entirety.

Although preferred embodiments of the invention have been described herein in detail, it will be understood by those skilled in the art that variations may be made thereto without departing from the spirit of the invention or the scope of the appended claims.

What is claimed is:

1. A VISTA agonist for inhibiting an immune response, wherein the VISTA agonist causes at least one of the following:
an increase in IL-10 expression;
a decrease in proliferation of CD4⁺ T cells;
a decrease in proliferation of CD8⁺ T cells;
a decrease in IL-2 expression;
a decrease in IFN-γ expression; and
a decrease in IL-17 expression.

2. The VISTA agonist of claim 1, wherein the VISTA agonist causes an increase in IL-10 expression, a decrease in proliferation of CD4⁺ and/or CD8⁺ T cells, and a decrease in IL-2 expression.

3. The VISTA agonist of claim 1 or 2, comprising or consisting of a polypeptide having the amino acid sequence of CDR1, CDR2, and/or CDR3 of Nb7, 7E12, 7G5, 3C3, 7C6, 7C7, 7G1, and/or 8G10:

| | CDR1 | CDR2 | CDR3 |
|---|---|---|---|
| Nb7 | AFTNAYMN | TRDSNRTS | AEPSGWWLDDDYW |
| | (SEQ ID NO: 1) | (SEQ ID NO: 2) | (SEQ ID NO: 3) |
| 7E12 | GYSITSGYY | ISYDGNN | ARGDYGSEAMDY |
| | (SEQ ID NO: 4) | (SEQ ID NO: 5) | (SEQ ID NO: 6) |
| 7G5 | GYTFTNYG | INTYTGEP | ARRSNPWPNDY |
| | (SEQ ID NO: 7) | (SEQ ID NO: 8) | (SEQ ID NO: 9) |
| 3C3 | GFNIRNTY | IDPANGNT | IMHAEGQGWFAY |
| | (SEQ ID NO: 10) | (SEQ ID NO: 11) | (SEQ ID NO: 12) |
| 7C6 | GYTFTSYY | INTGSGGT | AREGTFYSGDVGYWYFDF |
| | (SEQ ID NO: 13) | (SEQ ID NO: 14) | (SEQ ID NO: 15) |
| 7C7 | GFTFNKYW | ITNSGGNT | TLGVDY |
| | (SEQ ID NO: 16) | (SEQ ID NO: 17) | (SEQ ID NO: 18) |
| 7G1 | GLSLTSNS | IWSNGGT | ARYYDGSYYWYFDF |
| | (SEQ ID NO: 19) | (SEQ ID NO: 20) | (SEQ ID NO: 21) |
| 8G10 | GYTFTDYE | IDPETGGT | TRWSLRPYAMDY |
| | (SEQ ID NO: 22) | (SEQ ID NO: 23) | (SEQ ID NO: 24) |

or a functional variant or fragment thereof that binds to and agonizes VISTA.

4. The VISTA agonist of claim 3, comprising or consisting of a polypeptide having the amino acid sequence of the heavy and/or light chain of 7E12, 7G5, 3C3, 7C6, 7C7, 7G1, and/or 8G10 and/or the amino acid sequence of Nb7:
7E12 Heavy Chain:
(SEQ ID NO: 25)
DVHLQESGPGLVKPSQSLSLTCSVTGYSITSGYYWNWIRQFPGNKLEWMGYISYDGNNNYNPSLKN
RISITRDTSKNQFFLKLNSVTTEDTATYYCARGDYGSEAMDYWGQGTSVTVSS
7E12 Light Chain:
(SEQ ID NO: 26)
DIRVTQSPASLAVSLGQRATISCRASKSVSTSGYSYMNWYQQKPGQPPQLLIYLASNLESGVPARFS
GSGSGTDFTLNIHPVEEEDAAIYYCQHSRELPWTFGGGTKLEIK
7G5 Heavy Chain:
(SEQ ID NO: 27)
QIQLVQSGPELKKPGETVKISCKASGYTFTNYGMNWVKQAPEKGLKWMGWINTYTGEPTYADDFKG
RFAFSLETSASTAYLQINNLKNEDTATYFCARRSNPWPNDYWGQGTSLTVSS
7G5 Light Chain:
(SEQ ID NO: 28)
DIQMTQSPSSFSASLGERVSLTCRASQEISGYLSWLQQKPDGTIKRLIYAASTLDSGVPKRFSGSRSG
SDYSLTISSLESEDFADYYCLQYASYPWTFGGGTKLEIK
3C3 Heavy Chain:
(SEQ ID NO: 29)
EVKLQQSGAELGKPGASVKLSCKVSGFNIRNTYMHWVNQRPGKGLEWIGRIDPANGNTIYAEKFKSK
ATLTADTSSNTVYIQLSQLKSDDTAIYFCIMHAEGQGWFAYWGQGTLVTVSS
3C3 Light Chain:
(SEQ ID NO: 30)
IVLTQSPASLAVSLGQRATISYRASKSVSTSGYSYMHWNQQKPGQPPRLLIYLVSNLESGVPARFSGS
GSGTDFTLNIHPVEEEDAATYYCQHIRELTRSEGGPSWK
7C6 Heavy Chain:
(SEQ ID NO: 31)
QVQLQQSGAELAKPGSSVKISCKASGYTFTSYYISWIKQTTGQGLEYIGYINTGSGGTNYNEKFKGKA
TLTVDKSSSTAFMQLSSLTPDDSAVYYCAREGTFYSGDVGYWYFDFWGPGSMVTVSS
7C7 Heavy Chain:
(SEQ ID NO: 32)
EVQLVESGGGLVQPGRSLKLSCVASGFTFNKYWMSWTRQAPGKGLEWVASITNSGGNTYYPDSVK
GRFTISRDNAQNTLYLQMNSLRSEDTATYYCTLGVDYWGQGAMVTVSS
7C7 Light Chain:
(SEQ ID NO: 33)
DVVMTQTPPSLSVTIGQSVSISCKSSQSLVYSDGKTYLHWLLQSSGRSPKRLIYQVSNLASGVPDRFS
GTGSQKDFTLKISRVEAKDLGVYYCAQTTHEPYTFGAGTKLEMK
7G1 Heavy Chain:
(SEQ ID NO: 34)
QVQLKESGPGLVQPSQTLSLTCTVSGLSLTSNSVSWIRQPPGKGLEWMGAIWSNGGTDYNSAIKSR
LSISRDTSKSQVFLKVNSLQTEDAMYFCARYYDGSYYWYFDFWGPGTMVTVSS
7G1 Light Chain:
(SEQ ID NO: 35)
IVLTQSPASLAVSLGQRATISYRASKSVSTSGYSYMHWNQQKPGQPPRLLIYLVSNLESGVPARFSGS
GSGTDFTLNIHPVEEEDAATYYCQHIRELTRSEGGPSWK
8G10 Heavy Chain:
(SEQ ID NO: 36)
QVQLQQSGAELVRPGASVTLSCKASGYTFTDYEMHWVKQTPVHGLEWIGAIDPETGGTAYNQKFKG
KATLTADKSSSTAYMELRSLTSEDSAVYYCTRWSLRPYAMDYWGQGTSVTVSS
8G10 Light Chain:
(SEQ ID NO: 37)
DVVMTHTPLSLPVSLGDQASISCRSSQSLVHNNGNTYLHWYLQKPGQSPKLLIYKVSNRFSGVPDRF
SGSGSGTDFTLKISRVEAEDLGLYFCSQSTHVPWTFGGGTKLEIK
Nb7:
(SEQ ID NO: 38)
QVQLQESGGGLVYPGGSLRLSCTASGFAFTNAYMNWVRQAPGKGAEWVSGITRDSNRTSYADSVK
GRFTISRDNAKNTLYLQMDSLKSDDTALYYCNAEPSGWWLDDDYWGQGTQVTVSS
or a functional variant or fragment thereof that binds to and agonizes VISTA.

5. The VISTA agonist of claim 3 or 4, wherein the functional variant has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the recited amino acid sequence.

6. The VISTA agonist of claim 5, comprising or consisting of a polypeptide having 100% identity to the recited amino acid sequence.

7. A polynucleotide encoding the VISTA agonist of any one of claims 3 to 6.

8. A vector comprising the polynucleotide of claim 7.

9. A host cell comprising the vector of claim 8.

10. A composition comprising the VISTA agonist of any one of claims 1 to 6 and a carrier.

11. The VISTA agonist of any one of claims 1 to 6, the polynucleotide of claim 7, the vector of claim 8, the host cell of claim 9, or the composition of claim 10 for treating and/or preventing an inflammatory disorder.

12. The VISTA agonist of any one of claims 1 to 6, the polynucleotide of claim 7, the vector of claim 8, the host cell of claim 9, or the composition of claim 10 for treating and/or preventing an autoimmune disease.

13. A method for inhibiting an immune response, the method comprising administering the VISTA agonist of any one of claims 1 to 6, the polynucleotide of claim 7, the vector of claim 8, the host cell of claim 9, or the composition of claim 10.

14. A method for treating and/or preventing an inflammatory disorder, the method comprising administering the VISTA agonist of any one of claims 1 to 6, the polynucleotide of claim 7, the vector of claim 8, the host cell of claim 9, or the composition of claim 10.

15. A method for treating and/or preventing an autoimmune disease, the method comprising administering the VISTA agonist of any one of claims 1 to 6, the polynucleotide of claim 7, the vector of claim 8, the host cell of claim 9, or the composition of claim 10.

16. Use of the VISTA agonist of any one of claims 1 to 6, the polynucleotide of claim 7, the vector of claim 8, the host cell of claim 9, or the composition of claim 10 for inhibiting an immune response.

17. Use of the VISTA agonist of any one of claims 1 to 6, the polynucleotide of claim 7, the vector of claim 8, the host cell of claim 9, or the composition of claim 10 for treating and/or preventing an inflammatory disorder.

18. Use of the VISTA agonist of any one of claims 1 to 6, the polynucleotide of claim 7, the vector of claim 8, the host cell of claim 9, or the composition of claim 10 for treating and/or preventing an autoimmune disease.
